(19) United States
(12) Patent Application Publication
(10) Pub. No.: US 2020/0203016 A1
(43) Pub. Date: Jun. 25, 2020

(54) CANCER TISSUE SOURCE OF ORIGIN PREDICTION WITH MULTI-TIER ANALYSIS OF SMALL VARIANTS IN CELL-FREE DNA SAMPLES

(71) Applicant: GRAIL, Inc., Menlo Park, CA (US)

(72) Inventors: Earl Hubbell, Palo Alto, CA (US); Qinwen Liu, Fremont, CA (US)

(21) Appl. No.: 16/719,938

(22) Filed: Dec. 18, 2019

Related U.S. Application Data
(60) Provisional application No. 62/782,087, filed on Dec. 19, 2018.

(51) Int. Cl. G16H 50/20; G16H 10/40; G16H 70/60; G16H 50/70; G16H 10/60; G06N 5/04; G06N 5/00; G06N 20/10; G06N 20/20; G16B 30/00; G16B 20/00

(52) U.S. Cl. CPC G16H 50/20; G16H 10/40; G16H 70/60; G16H 50/70; G16H 10/60; G16B 20/00; G06N 5/003; G06N 20/10; G06N 20/20; G16B 30/00; G06N 5/04

(57) ABSTRACT

A predictive cancer model generates a prediction of cancer tissue source of origin for a subject of interest by analyzing values of one or more types of features that are derived from cfDNA obtained from the individual. Specifically, cfDNA from the individual is sequenced to generate sequence reads using one or more physical assays, examples of which include a small variant sequencing assay. The sequence reads of the physical assays are processed through corresponding computational analyses to generate small variant features and other features. The values of features can be provided to a prediction model that generates a prediction of cancer tissue source of origin and/or cancer presence.

### CROSS-REFERENCE TO RELATED APPLICATIONS

This application claims the benefit of priority to U.S. Provisional Patent Application No. 62/782,087, filed on Dec. 19, 2018, and entitled “CANCER TISSUE SOURCE OF ORIGIN PREDICTION WITH MULTI-TIER ANALYSIS OF SMALL VARIANTS IN CELL-FREE DNA SAMPLES,” the contents of which is herein incorporated by reference in its entirety.

### TECHNICAL FIELD

This disclosure generally relates to predicting a cancer tissue source of origin in a subject, and more specifically to performing one or more physical and/or computational assays on a test sample obtained from a subject in order to predict cancer tissue source of origin.

### BACKGROUND

Analysis of circulating cell-free nucleotides, such as cell-free DNA (cfDNA), using next generation sequencing (NGS) is recognized as a valuable tool for detection and diagnosis of cancer. Analyzing cfDNA can be advantageous in comparison to traditional tumor biopsy methods; however, identifying in tumor-derived cfDNA faces distinct challenges, especially for purposes such as early detection of cancer and early predictions of cancer tissue source of origin, where the cancer-indicative signals are not yet pronounced. Various challenges stand in the way of accurately predicting, with sufficient sensitivity and specificity, characteristics of and sources of cancers in subjects through the use of cfDNA.

### SUMMARY

Embodiments described provide for a method of generating a prediction of a cancer tissue of origin, in addition to generating a prediction of presence or absence of cancer, for one or more subjects based on cfDNA in test sample(s) obtained from the subject(s). As such, the invention can be used to resolve tissue of origin for a cancer, in addition to generating predictions for detection of cancer presence in one or more subjects.

Specifically, cfDNA from the subject(s) is sequenced to generate sequence reads using one or more sequencing assays, also referred to herein as physical assays, an example of which includes a small variant sequencing assay. The sequence reads of the physical assays are processed through corresponding computational analyses, where computational assays and/or physical assays are used to extract features including small variant features and/or copy number features. The physical and computational analyses thus output values of features of sequence reads that are informative for generating predictions of cancer tissue source of origin. As examples, small variant features (e.g., features derived from sequence reads that were generated by a small variant sequencing assay) can include a total number of somatic variants, and copy number features can include focal copy number. Additional features that are not derived from sequencing-based approaches, such as baseline features that can refer to clinical symptoms and patient information, can be further generated and analyzed.

In some embodiments, one or more features or types of types of features (e.g., small variant features, copy number features, etc.) can be provided to a predictive model that generates a prediction of cancer tissue source of origin and/or a prediction of presence of cancer. In some embodiments, the values of different features and/or types of features can be separately provided into different predictive models. Each separate predictive model can output a score that then serves as input into an overall model that outputs the cancer prediction.

Embodiments disclosed herein describe a method for determining a cancer tissue of origin for a subject, the method including: accessing, upon processing a cell-free deoxyribonucleic acid (cfDNA) sample from the subject, a dataset comprising sequence reads generated from application of a physical assay to the cfDNA sample; performing a computational assay on the dataset to generate values of a set of features; processing the set of features with a prediction model to generate a prediction of a cancer tissue of origin for the subject from a set of candidate tissue sources, the prediction model transforming the values of the set of features into the prediction through a function; and returning the prediction of the tissue source of origin related to presence of cancer in the subject. In some embodiments, the method determines confidences in outputted predictions and provides the predictions to relevant entities based on the confidences.

In some embodiments, the prediction model is a multi-tiered model that classifies the subject into a cancerous group or a non-cancerous group in a first sub-model, and that generates the prediction of tissue source of origin upon application of a second sub-model. In some embodiments, the first sub-model is a binomial classification model. In some embodiments, the second sub-model is a multinomial regression model (e.g., penalized multinomial regression model). However, in alternative embodiments, the first sub-model and/or the second sub-model can include other model architectures.

In some embodiments, the method predicts the tissue source of origin related to presence of cancer from candidate tissue sources of origin including one or more of: a uterine tissue source, a thyroid tissue source, a renal tissue source, a prostate tissue source, a pancreas tissue source, an ovarian tissue source, a multiple myeloma tissue source, a lymphoma tissue source, a lung tissue source, a leukemia tissue source, a hepatobiliary tissue source, a head tissue source, a neck tissue source, a gastric tissue source, an esophageal tissue source, a colorectal tissue source, a cervical tissue source, a breast tissue source, and a bladder tissue source, another tissue source, and any combination or grouping of tissue sources (e.g., female reproductive system tissue sources, head and neck tissue sources, gastrointestinal tissue sources, etc.).

In some embodiments, the subject is asymptomatic. In some embodiments, the cell-free nucleic acids comprise cell-free DNA (cfDNA). In some embodiments, the sequence reads are generated from a next generation sequencing (NGS) procedure. In some embodiments, the sequence reads are generated from a massively parallel sequencing procedure using sequencing-by-synthesis.

In some embodiments, the test sample is a blood, plasma, serum, urine, cerebrospinal fluid, fecal matter, saliva, pleural fluid, pericardial fluid, cervical swab, saliva, or peritoneal fluid sample.

### BRIEF DESCRIPTION OF THE DRAWINGS

FIG. 1A depicts an overall flow process for generating a prediction of the tissue source of origin related to presence of cancer based on features derived from a cfDNA sample obtained from a subject, in accordance with one or more embodiments.

FIG. 1B depicts an overall flow diagram for determining a prediction of the tissue source of origin related to presence of cancer using at least a cfDNA sample obtained from a subject, in accordance with one or more embodiments.

FIG. 1C depicts a variation of FIG. 1B that utilizes sub-models for determining a prediction of the tissue source of origin related to presence of cancer using at least a cfDNA sample obtained from a subject, in accordance with one or more embodiments.

FIG. 1D depicts an overall flow diagram for determining a prediction of the tissue source of origin and/or other prediction based on various input features and sub-models, in accordance with one or more embodiments.

FIG. 1E depicts an overall flow diagram for determining a prediction of the tissue source of origin based on multiple types of input features that are processed separately by multiple prediction models, in accordance with one or more embodiments.

FIG. 2A depicts a flow process of a method for performing a sequencing assay to generate sequence reads, in accordance with one or more embodiments.

FIG. 2B depicts a variation of FIG. 2A for performing a sequencing assay to generate sequence reads, in accordance with one or more embodiments.

FIG. 3A is an example flow process for performing a data workflow to analyze sequence reads generated by a small variant sequencing assay, in accordance with one or more embodiments.

FIG. 3B depicts a flow process for generating feature vectors as inputs to a prediction model, with application of a quality criterion, in accordance with one or more embodiments.

FIG. 4A depicts an example of a model architecture for processing a feature vector to predict tissue source of origin, in accordance with one or more embodiments.

FIG. 4B depicts an embodiment of model coefficient outputs for features associated with different genes, in relation to predictions of tissue sources of origin in accordance with one or more embodiments.

FIG. 4C depicts a flow process for applying an embodiment of a prediction model to a feature vector derived from a sample from a subject, to return a tissue source of origin prediction, in accordance with one or more embodiments.

FIG. 5A depicts an example of precision metric outputs of a predictive model, in relation to predictions of the tissue sources of origin shown in TABLES 1-22, in accordance with one or more embodiments.

FIG. 5B depicts an example of recall metric outputs of a predictive model, in relation to predictions of the tissue sources of origin shown in TABLES 1-22, in accordance with one or more embodiments.

FIG. 6A depicts an example of model coefficient outputs for features associated with different genes, in relation to a prediction of a breast tissue source of origin, in accordance with one or more embodiments.

FIG. 6B depicts an example of model coefficient outputs for features associated with different genes, in relation to a prediction of a colorectal tissue source of origin, in accordance with one or more embodiments.

FIG. 6C depicts an example of model coefficient outputs for features associated with different genes, in relation to a prediction of a lung tissue source of origin, in accordance with one or more embodiments.

FIG. 6D depicts an example of model coefficient outputs for features associated with different genes, in relation to a prediction of a non-cancer grouping, in accordance with one or more embodiments.

FIG. 6E depicts an example of model coefficient outputs for features associated with different genes, in relation to a prediction of a pancreas tissue source of origin, in accordance with one or more embodiments.

FIG. 6F depicts an example of model coefficient outputs for features associated with different genes, in relation to a prediction of a bladder tissue source of origin, in accordance with one or more embodiments.

FIG. 6G depicts an example of model coefficient outputs for features associated with different genes, in relation to a prediction of a cancer of unknown primary tissue source of origin, in accordance with one or more embodiments.

FIG. 6H depicts an example of model coefficient outputs for features associated with different genes, in relation to a prediction of a cervix tissue source of origin, in accordance with one or more embodiments.

FIG. 6I depicts an example of model coefficient outputs for features associated with different genes, in relation to a prediction of an esophogeal tissue source of origin, in accordance with one or more embodiments.

FIG. 6J depicts an example of model coefficient outputs for features associated with different genes, in relation to a prediction of a gastric tissue source of origin, in accordance with one or more embodiments.

FIG. 6K depicts an example of model coefficient outputs for features associated with different genes, in relation to a prediction of a head/neck tissue source of origin, in accordance with one or more embodiments.

FIG. 6L depicts an example of model coefficient outputs for features associated with different genes, in relation to a prediction of a hepatobiliary tissue source of origin, in accordance with one or more embodiments.

FIG. 6M depicts an example of model coefficient outputs for features associated with different genes, in relation to a prediction of a lymphoma tissue source of origin, in accordance with one or more embodiments.

FIG. 6N depicts an example of model coefficient outputs for features associated with different genes, in relation to a prediction of a melanoma tissue source of origin, in accordance with one or more embodiments.

FIG. 6O depicts an example of model coefficient outputs for features associated with different genes, in relation to a prediction of a multiple myeloma tissue source of origin, in accordance with one or more embodiments.

FIG. 6P depicts an example of model coefficient outputs for features associated with different genes, in relation to a prediction of an other tissue source of origin, in accordance with one or more embodiments.

FIG. 6Q depicts an example of model coefficient outputs for features associated with different genes, in relation to a prediction of an ovarian tissue source of origin, in accordance with one or more embodiments.

FIG. 6R depicts an example of model coefficient outputs for features associated with different genes, in relation to a prediction of a prostate tissue source of origin, in accordance with one or more embodiments.

FIG. 6S depicts an example of model coefficient outputs for features associated with different genes, in relation to a prediction of a renal tissue source of origin, in accordance with one or more embodiments.

FIG. 6T depicts an example of model coefficient outputs for features associated with different genes, in relation to a prediction of a thyroid tissue source of origin, in accordance with one or more embodiments.

FIG. 6U depicts an example of model coefficient outputs for features associated with different genes, in relation to a prediction of a uterine tissue source of origin, in accordance with one or more embodiments.

FIG. 7 depicts an example computer system for implementing various methods of the present invention.

### DETAILED DESCRIPTION

The figures and the following description relate to preferred embodiments by way of illustration only. It should be noted that from the following discussion, alternative embodiments of the structures and methods disclosed herein will be readily recognized as viable alternatives that can be employed without departing from the principles of what is claimed.

Reference will now be made in detail to several embodiments, examples of which are illustrated in the accompanying figures. It is noted that wherever practicable similar or like reference numbers can be used in the figures and can indicate similar or like functionality. For example, a letter after a reference numeral, such as “prediction model 160a,” indicates that the text refers specifically to the element having that particular reference numeral. A reference numeral in the text without a following letter, such as “prediction model 160,” refers to any or all of the elements in the figures bearing that reference numeral (e.g. “prediction model 160” in the text refers to reference numerals “prediction model 160a” and/or “prediction model 160b” in the figures).

The term “individual” refers to a human individual. The term “healthy individual” refers to an individual presumed to not have a cancer or disease. The term “subject” refers to an individual who is known to have, or potentially has, a cancer or disease.

The term “sequence reads” refers to nucleotide sequences read from a sample obtained from an individual. Sequence reads can be obtained through various methods known in the art.

The term “read segment” or “read” refers to any nucleotide sequences including sequence reads obtained from an individual and/or nucleotide sequences derived from the initial sequence read from a sample obtained from an individual. For example, a read segment can refer to an aligned sequence read, a collapsed sequence read, or a stitched read. Furthermore, a read segment can refer to an individual nucleotide base, such as a single nucleotide variant.

The term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y can be denoted as “X>Y.” For example, a cytosine to thymine SNV can be denoted as “C>T.”

The term “indel” refers to any insertion or deletion of one or more bases having a length and a position (which can also be referred to as an anchor position) in a sequence read. An insertion corresponds to a positive length, while a deletion corresponds to a negative length.

The term “mutation” refers to one or more SNVs or indels.

The term “candidate variant,” “called variant,” or “putative variant” refers to one or more detected nucleotide variants of a nucleotide sequence, for example, at a position in the genome that is determined to be mutated (i.e., a candidate SNV) or an insertion or deletion at one or more bases (i.e., a candidate indel). Generally, a nucleotide base is deemed a called variant based on the presence of an alternative allele on a sequence read, or collapsed read, where the nucleotide base at the position(s) differ from the nucleotide base in a reference genome. Additionally, candidate variants can be called as true positives or false positives.

The term “true positive” refers to a mutation that indicates real biology, for example, presence of a potential cancer, disease, or germline mutation in an individual. True positives are not caused by mutations naturally occurring in healthy individuals (e.g., recurrent mutations) or other sources of artifacts such as process errors during assay preparation of nucleic acid samples.

The term “false positive” refers to a mutation incorrectly determined to be a true positive. Generally, false positives can be more likely to occur when processing sequence reads associated with greater mean noise rates or greater uncertainty in noise rates.

The term “cell-free nucleic acids” of “cfNAs” refers to nucleic acid molecules that can be found outside cells, in bodily fluids such blood, sweat, urine, or saliva. Cell-free nucleic acids are used interchangeably as circulating nucleic acids.

The term “cell-free deoxyribonucleic acid,” “cell free DNA,” or “cfDNA” refers to deoxyribonucleic acid fragments that circulate in bodily fluids such blood, sweat, urine, or saliva and originate from one or more healthy cells and/or from one or more cancer cells.

The term “circulating tumor DNA” or “ctDNA” refers to deoxyribonucleic acid fragments that originate from tumor cells or other types of cancer cells, which can be released into an individual's bodily fluids such blood, sweat, urine, or saliva as result of biological processes such as apoptosis or necrosis of dying cells or actively released by viable tumor cells.

The term “circulating tumor RNA” or “ctRNA” refers to ribonucleic acid fragments that originate from tumor cells or other types of cancer cells, which can be released into an individual's bodily fluids such blood, sweat, urine, or saliva as result of biological processes such as apoptosis or necrosis of dying cells or actively released by viable tumor cells.

The term “genomic nucleic acid,” “genomic DNA,” or “gDNA” refers to nucleic acid including chromosomal DNA that originate from one or more healthy cells.

The term “alternative allele” or “ALT” refers to an allele having one or more mutations relative to a reference allele, e.g., corresponding to a known gene.

The term “sequencing depth” or “depth” refers to a total number of read segments from a sample obtained from an individual at a given position, region, or loci. In some embodiments, the depth refers to the average sequencing depth across the genome or across a targeted sequencing panel.

The term “alternate depth” or “AD” refers to a number of read segments in a sample that support an ALT, e.g., include mutations of the ALT.

The term “reference depth” refers to a number of read segments in a sample that include a reference allele at a candidate variant location.

The term “alternate frequency” or “AF” refers to the frequency of a given ALT. The AF can be determined by dividing the corresponding AD of a sample by the depth of the sample for the given ALT.

The term “variant” or “true variant” refers to a mutated nucleotide base at a position in the genome. Such a variant can lead to the development and/or progression of cancer in an individual.

The term “edge variant” refers to a mutation located near an edge of a sequence read, for example, within a threshold distance of nucleotide bases from the edge of the sequence read.

The term “non-edge variant” refers to a candidate variant that is not determined to be resulting from an artifact process, e.g., using an edge variant filtering method described herein. In some scenarios, a non-edge variant may not be a true variant (e.g., mutation in the genome) as the non-edge variant could arise due to a different reason as opposed to one or more artifact processes.

The term “copy number aberrations” or “CNAs” refers to changes in copy number in somatic tumor cells. For example, CNAs can refer to copy number changes in a solid tumor.

The term “copy number variations” or “CNVs” refers to changes in copy number changes that derive from germline cells or from somatic copy number changes in non-tumor cells. For example, CNVs can refer to copy number changes in white blood cells that can arise due to clonal hematopoiesis.

The term “copy number event” refers to one or both of a copy number aberration and a copy number variation.

### 1. Generating a Cancer Prediction

1.1. Overall Process Flow

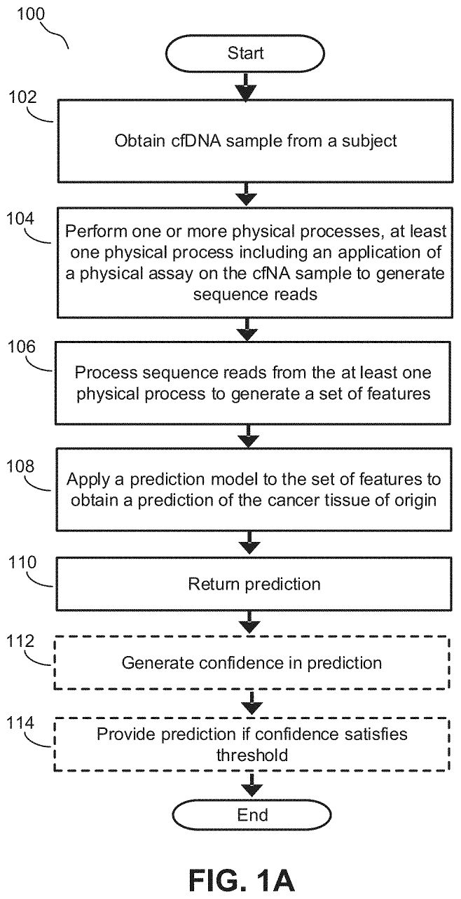

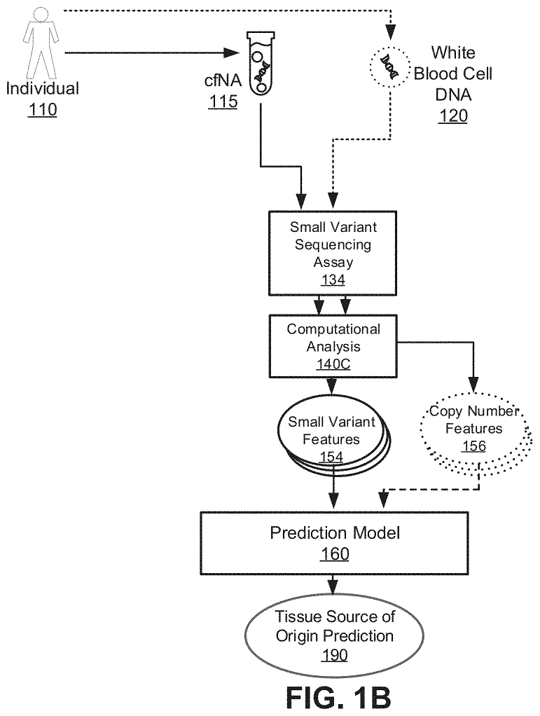

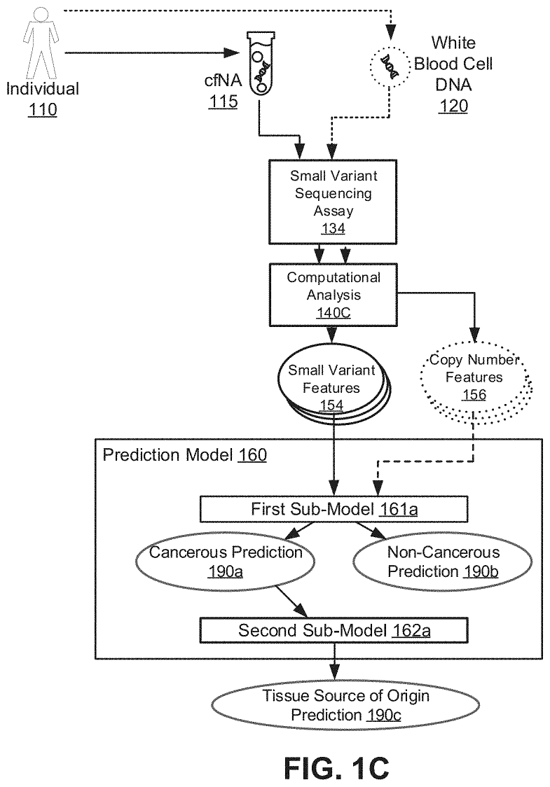

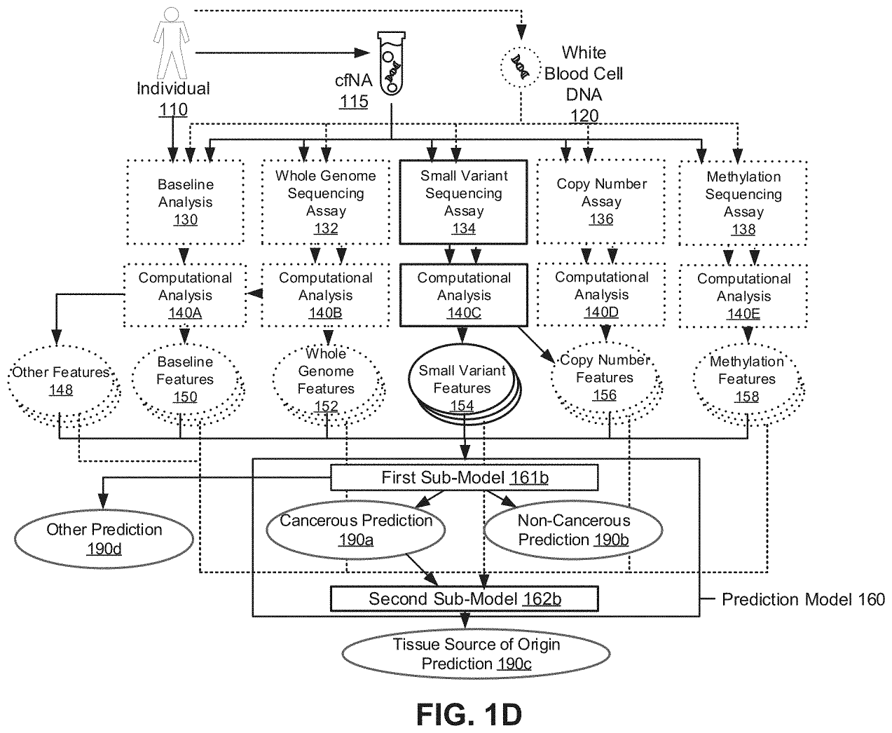

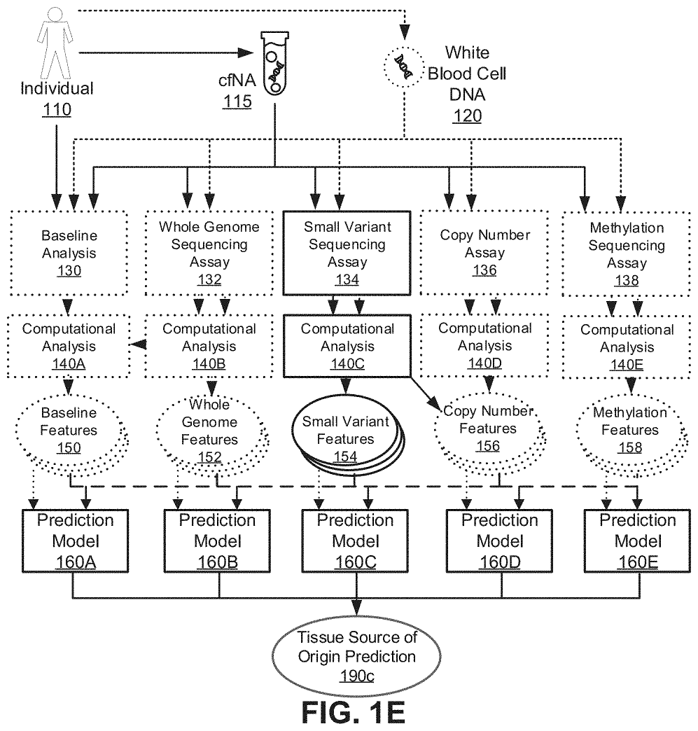

FIG. 1A depicts an overall flow process 100 for generating a prediction of a cancer tissue source of origin based on features derived from a cfDNA sample obtained from an individual, in accordance with an embodiment. Further reference will be made to FIGS. 1B-1E, each of which depicts an overall flow diagram for determining a cancer prediction using at least a cfDNA sample obtained from an individual, in accordance with an embodiment.

At step 102, the test sample is obtained from the individual (e.g., from a sampling device, from automated sampling equipment). Generally, samples can be from healthy subjects, subjects known to have or suspected of having cancer, or subjects where no prior information is known (e.g., asymptomatic subjects). The test sample can be a sample of one or more of: blood, plasma, serum, urine, fecal, and saliva samples. Alternatively, the test sample can include a sample of one or more of: whole blood, a blood fraction, a tissue biopsy, pleural fluid, pericardial fluid, cerebral spinal fluid, and peritoneal fluid.

As shown in each of FIGS. 1B-1E, a test sample can include cfDNA 115. In various embodiments, a test sample can additionally or alternatively include genomic DNA (gDNA). An example of a source of gDNA, as shown in FIGS. 1B-1E, is white blood cell (WBC) DNA 120.

At step 104, one or more physical process analyses are performed (e.g., by laboratory apparatus including a sequencing system), where at least one physical process analysis includes a sequencing-based assay on cfDNA 115 to generate sequence reads. Referring to FIGS. 1B-1C, examples of a physical process analysis can include a small variant sequencing assay 134. Referring to FIGS. 1D-1E, additional physical process analyses can include one or more of: a baseline analysis 130, a whole genome sequencing assay 132, a copy number assay 136, and a methylation sequencing assay 138.

A small variant sequencing assay refers to a physical assay that generates sequence reads, typically through targeted gene sequencing panels that can be used to determine small variants, examples of which include single nucleotide variants (SNVs) and/or insertions or deletions. Alternatively, assessment of small variants can also be done using a whole genome sequencing approach or a whole exome sequencing approach. As described below, and in relation to FIGS. 1C, 1D, and 1E, outputs of the small variant sequencing assay 134, with performance of a computational analysis 140C, can be used to generate small variant features and/or copy number features 156, with or without performance of the copy number assay described in relation to FIGS. 1D and 1E. In examples, the computational analysis can involve any number of trained models (“Bayesian Hierarchical model,” “Joint Model,” etc.) or filters of the embodiments described herein.

A baseline analysis 130 of the individual 110 can include a clinical analysis of the individual 110 and can be performed by a physician or a medical professional. In some embodiments, the baseline analysis 130 can include an analysis of germline changes detectable in the cfDNA 115 of the individual 110. In some embodiments, the baseline analysis 130 can perform the analysis of germline changes with additional information such as an identification of upregulated or downregulated genes. Such additional information can be provided by a computational analysis, such as computational analysis 140A as depicted in FIGS. 1D-1E. The baseline analysis 130 is described in further detail below.

A whole genome sequencing assay refers to a physical assay that generates sequence reads for a whole genome or a substantial portion of the whole genome. Such a physical assay can employ whole genome sequencing techniques or whole exome sequencing techniques.

A copy number assay refers to a physical assay that generates, from sequence reads, outputs describing larger scale variations (or variations across longer sequences), such as copy number variations or copy number aberrations. Such a physical assay can employ whole genome or whole exome sequencing techniques, or other sequencing techniques operable to acquire copy number variation characteristics of a sample.

A methylation sequencing assay refers to a physical assay that generates sequence reads which can be used to determine the methylation status of a plurality of CpG sites, or methylation patterns, across the genome. An example of such a methylation sequencing assay can include the bisulfate treatment of cfDNA for conversion of unmethylated cytosines (e.g., CpG sites) to uracil (e.g., using EZ DNA Methylation-Gold or an EZ DNA Methylation-Lightning kit (available from Zymo Research Corp)). Alternatively, an enzymatic conversion step (e.g., using a cytosine deaminase (such as APOBEC-Seq (available from NEBiolabs))) can be used for conversion of unmethylated cytosines to uracils. Following conversion, the converted cfDNA molecules can be sequenced through a whole genome sequencing process or a targeted gene sequencing panel and sequence reads used to assess methylation status at a plurality of CpG sites. Methylation-based sequencing approaches are known in the art (e.g., see US 2014/0080715, which is incorporated herein by reference). In another embodiment, DNA methylation can occur in cytosines in other contexts, for example CHG and CHH, where H is adenine, cytosine or thymine. Cytosine methylation in the form of 5-hydroxymethylcytosine can also be assessed (see, e.g., WO 2010/037001 and WO 2011/127136, which are incorporated herein by reference), and features thereof, using the methods and procedures disclosed herein. In some embodiments, a methylation sequencing assay need not perform a base conversion step to determine methylation status of CpG sites across the genome. For example, such methylation sequencing assays can include PacBio sequencing or Oxford Nanopore sequencing.

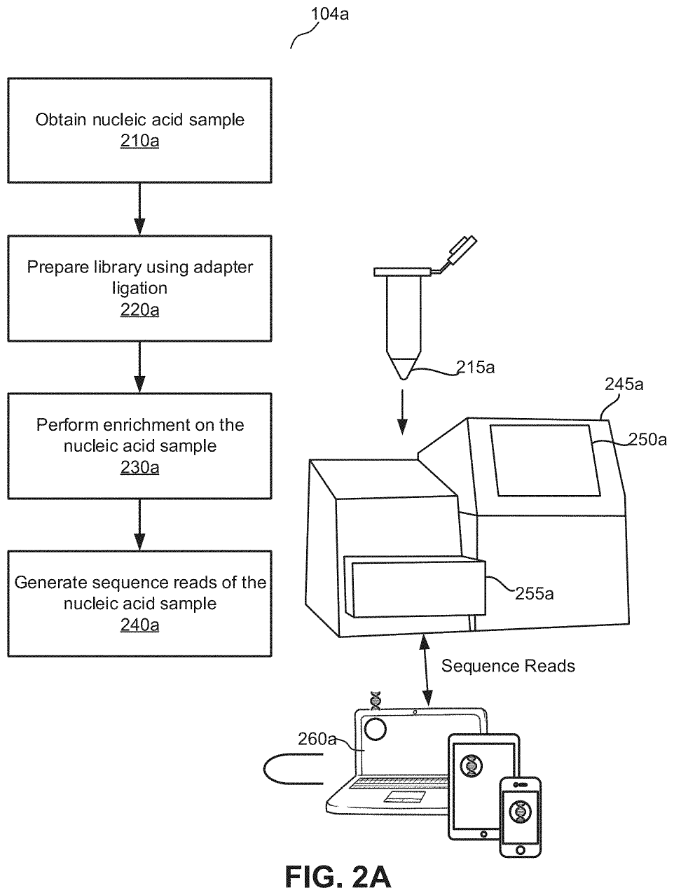

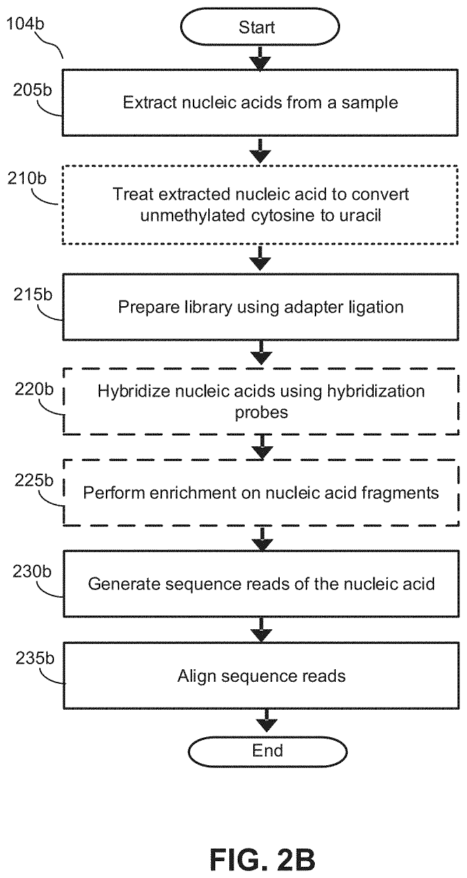

The small variant sequencing assay 134 and/or other assays are performed by respective system components on the cfDNA 115 to generate and process sequence reads of the cfDNA 115. In various embodiments, the small variant sequencing assay 134 and/or one or more of the whole genome sequencing assay 132, copy number assays 136, and methylation sequencing assay 138 can be further performed by respective system components on the WBC DNA 120 to generate sequence reads of the WBC DNA 120. The process steps performed in each assay are described in further detail in relation to FIG. 2.

At step 106, the sequence reads generated as a result of performing the sequencing-based assay are processed to determine values for features. Features, generally, are types of information obtainable from physical assays and/or computational analyses that can be used in predicting tissue source of origin for a cancer and/or presence of cancer in a subject. Generally, the predictions for identifying tissue source of origin and/or cancer presence in an individual are based on transformation of input features, as constituent components of one or more model architectures, into predictive outputs.

Sequence reads are processed by applying one or more computational analyses, described in more detail in relation to FIGS. 1B-1E. Generally, each computational analysis 140 represents an algorithm that is executable by a processor of a computer, hereafter referred to as a processing system. Therefore, each computational analysis analyzes sequence reads and outputs values features based on the sequence reads. Each computational analysis is specific for a given sequencing-based assay and therefore, each computational analysis outputs a particular type of feature that is specific for the sequencing-based assay.

As shown in FIGS. 1B-1E, sequence reads generated from application of a small variant sequencing assay are processed using a computational analysis 140C, otherwise referred to as a small variant computational analysis. The computational analysis 140C outputs small variant features 154. Additionally or alternatively, sequence reads generated from application of a whole genome sequencing assay 132 are processed using computational analysis 140B, otherwise referred to as a whole genome computational analysis. The computational analysis 140B outputs whole genome features 152. Additionally or alternatively, sequence reads generated from application of a copy number assay 136 are processed using computational analysis 140D, otherwise referred to as a copy number computational analysis. The computational analysis 140D outputs copy number features 156 (which can also be output by the computational analyses 140C). Additionally or alternatively, sequence reads generated from application of a methylation sequencing assay are processed using computational analysis 140E, otherwise referred to as a methylation computational analysis. The computational analysis 140E outputs methylation features 158. Additionally or alternatively, computational analysis 140A analyzes information from the baseline analysis 130 and outputs baseline features 150.

At step 108, a prediction model is applied to the features to generate a prediction of the tissue source of origin related to presence of cancer for the individual 110. Examples of the prediction of the tissue source of origin include a prediction of one or more of: a uterine tissue source, a thyroid tissue source, a renal tissue source, a prostate tissue source, a pancreas tissue source, an ovarian tissue source, a multiple myeloma tissue source, a lymphoma tissue source, a lung tissue source, a leukemia tissue source, a hepatobiliary tissue source, a head tissue source, a neck tissue source, a gastric tissue source, an esophageal tissue source, a colorectal tissue source, a cervical tissue source, a breast tissue source, and a bladder tissue source. Examples of the prediction of the cancer tissue source can additionally or alternatively include predictions of a group of tissue sources for cancer origin in the subject(s), including one or more of: a grouping of gastrointestinal tissue sources (e.g., including gastric tissue, including esophageal tissue, etc.), female reproductive system tissue sources (e.g., including ovarian tissue, including breast tissue, including cervical tissue, etc.), male reproductive system tissue sources (e.g., including prostate tissue, etc.), head and neck tissue sources (e.g., including head tissues, including neck tissues, etc.), circulatory system tissue sources, neurological tissue sources (e.g., brain tissue, spinal cord tissue, etc.), and other groupings. Additionally or alternatively, the prediction model can, at different stages of generating a prediction, outputs indicating a presence or absence of cancer, a severity, stage, a grade of cancer, a cancer sub-type, a treatment decision, and a likelihood of response to a treatment, as described in more detail below.

In various embodiments, the prediction output of the prediction model is a score, such as a likelihood or probability, with a confidence value, that indicates a tissue of origin of cancer in the subject. The prediction output can additionally or alternatively include scores, with confidence values, for predictions of one or more of: a presence or absence of cancer, a severity, stage, a grade of cancer, a cancer sub-type, a treatment decision, and a likelihood of response to a treatment. Scores can be singular in characterizing presence/absence of cancer from a particular tissue source, characterizing a presence/absence of cancer from a grouping of tissue sources, or characterizing presence/absence of cancer generally. Alternatively, such scores can be plural, such that the output of the prediction model can include scores characterizing, for each of a set categories (e.g., of tissue sources, of groupings of tissue sources, of cancer presence, of cancer non-presences, etc.) a score, with a confidence value, for each category. For clarity of description, the output(s) of the prediction model are generally referred to as a set of scores, the set comprising one or more scores depending upon what the prediction model is configured to determine.

At step 110, the system returns the output(s) of the prediction model, with associated confidence values 112 associated with each prediction output. At step 114, the system then provides the output(s) of the prediction model if confidence(s) of the respective output(s) satisfies(y) a threshold condition. In some embodiments, the method can further include generating a value of a confidence parameter for an output of the prediction model and, upon determining satisfaction of a threshold condition by the value, providing the prediction to an entity (e.g., healthcare provider, etc.) for provision care to the user in relation to a prediction of cancer tissue source of origin and/or cancer presence.

The structure of the prediction model can be configured according to the particular features input into the prediction model, and/or according to outputs of the prediction model provided at different stages of generating a prediction, as described in more detail in relation to FIGS. 1B-1D below. Each particularly structured prediction model is described hereafter in relation to a processing workflow that generates values of one or more types of features that the prediction model receives. As used hereafter, a workflow process refers to the performance of the physical process analysis, computational analysis, and application of a predictive cancer model.

In an embodiment, as shown in FIG. 1B, the prediction model 160 can receive a first type of input feature, such as small variant features 154, and output a tissue source of origin prediction 190. Additionally, the prediction model 160 can receive a second type of input feature, such as copy number features 156 and, upon processing at least one of the small variant features 154 and the copy number features 156, output a tissue source of origin prediction 190.

As shown in FIG. 1C, in a variation of the embodiment shown in FIG. 1B, the prediction model can be constructed with multiple sub-models. In the embodiment shown in FIG. 1C, the prediction model includes a first sub-model 161a that receives one or more of the small variant features 154 and copy number features 156 as inputs, and outputs a prediction score associated with the subject belonging to a cancerous group 190a or a non-cancerous group 190b. The first sub-model 161a can also output a prediction score associated with an indeterminate prediction. The prediction model also includes a second sub-model 162a that, based on the small variant features 154, the copy number features 156, and/or outputs of the first sub-model 161a, outputs one or more predictions indicating cancer tissue source of origin 190c for the subject.

As such, as shown in FIG. 1C, the prediction model can group the subject into one of a cancerous group 190a and a non-cancerous group upon applying a first sub-model 161a of the prediction model, and upon determining that the subject is grouped into the cancerous group, apply a second sub-model 162b of the prediction model to generate the prediction of the cancer tissue of origin 190c for the subject. However, in variations of the embodiment shown in FIG. 1C, the prediction model can apply the second sub-model 162 without relying upon outputs of the first sub-model 161 and/or apply the sub-models in any other suitable order. Furthermore, in some examples, the same features used as inputs to the first sub-model 161a are also used as inputs to the second sub-model 162a. Additional and/or alternative features can be derived from the cfDNA sample using computational analysis as input to the second sub-model 162a. In some cases, the additional and/or alternative features are derived subsequent to and/or in accordance with a determination that the subject is grouped into the cancerous group 190a.

In the embodiment shown in FIG. 1D, the prediction model can be constructed to receive other types of input features, such as the baseline features 150, whole genome features 152, small variant features 154, methylation features 156, and/or other features 148 described briefly above. Similar to the embodiment shown in FIG. 1C, the prediction model in the embodiment shown in FIG. 1D includes a first sub-model 161b that receives one or more of the baseline features 150, whole genome features 152, small variant features 154, copy number features 156, methylation features 158, and other features 148 as inputs, and outputs a prediction score associated with the subject belonging to a cancerous group 190a or a non-cancerous group 190b. The first sub-model 161b can also output a prediction score associated with an indeterminate prediction. The prediction model also includes a second sub-model 162b that, based on the baseline features 150, whole genome features 152, small variant features 154, copy number features 156, methylation features 158, and other features 148, and/or outputs of the first sub-model 161b, outputs one or more predictions indicating cancer tissue source of origin 190c for the subject. As such, as shown in FIG. 1D, the prediction model can group the subject into one of a cancerous group 190a and a non-cancerous group 190b upon applying a first sub-model 161b of the prediction model, and upon determining that the subject is grouped into the cancerous group, apply a second sub-model 162b of the prediction model to generate the prediction of the cancer tissue of origin 190c for the subject. However, in variations of the embodiment shown in FIG. 1D, the prediction model can apply the second sub-model 162b without relying upon outputs of the first sub-model 161b and/or apply the sub-models in any other suitable order. Furthermore, in some examples, the same features used as inputs to the first sub-model 161b are also used as inputs to the second sub-model 162b. Additional and/or alternative features can be derived from the cfDNA sample using computational analysis as input to the second sub-model 162b. In some cases, the additional and/or alternative features are derived subsequent to a determination that the subject is grouped into the cancerous group 190a.

Furthermore, as shown in FIG. 1D, the system can, based upon an output of the first sub-model 161b, generate another prediction 190d associated with a health state of the subject and/or perform additional assays on the sample(s) from the subject. For instance, based upon an output of the first sub-model 161b, the system can perform a reflex assay on a reserve sample from the subject. Based upon the reflex assay, the system can then generate another prediction of a health state of the subject and/or output a prediction, with increased confidence, of a grouping of the subject into one of the cancerous group and the non-cancerous group (e.g., based on implementation of another sequencing-based assay). Merely by way of example, the baseline analysis 130 on the individual (e.g., on the individual's blood sample) can provide various clinical symptoms and/or patient information that can be used to corroborate with the cancer predictions from the prediction model 160 and/or used to provide features for input to the prediction model 160 to generate the cancer predictions or other predictions 190d. For instance, the individual's blood sample can be used for a complete blood count (“CBC”) that measures several components and features (e.g., non-sequencing-based features) in the individual's blood. Some features can include a WBC count, which can be used to augment the prediction of leukemia from the prediction model 160 when the WBC count is high, and/or a platelet count, which can be used to augment the prediction of liver cancer or liver failure when the platelet count is low, or other liver disease prediction 190d.

As shown in FIG. 1D, copy number features 156 can be extracted upon performing computational analyses 140c with outputs of the small variant sequencing assay 134 described above. Copy number features 156 can additionally or alternatively be extracted upon performing a computational analysis 140D on outputs of a copy number assay 136 performed on the sample(s) from the subject, in relation to other physical and/or computational assays.

In some embodiments, as shown in FIG. 1E, the system can include architecture for application of separate predictive cancer models, each structured to process one type of input feature. In this embodiment, at a first stage, the values of features output from each computational analysis (i.e., computational analyses 140A-140E) are separately input into individual sub-models (160A-160E) associated with each feature type. Then, the output of each individual sub-model is used to generate a tissue source of origin prediction 190c for a subject. In more detail, as shown in FIG. 1E, one or more of: baseline features 150 are provided as inputs to prediction model 160A, whole genome features 152 are provided as inputs to prediction model 160B, small variant features 154 are provided as inputs to prediction model 160C, copy number features 156 are provided as inputs to prediction model 160D, and methylation features 158 are provided as inputs to prediction model 160E. The output of each of predictive models 160A-160E can then be co-processed to generate a tissue source of origin prediction 190c for a subject.

Although FIG. 1E depicts that the output of five separate prediction models 160A-160E are used to generate a tissue source of origin prediction 190c for a subject, in various embodiments, additional or fewer prediction models can be involved in generating the tissue source of origin prediction 190c. For example, in some embodiments, any one, two, three, four, or five of the prediction models 160A-160E, with any other suitable prediction model configured to process other input features, can be used to output information for generating a tissue source of origin prediction 190c.

Furthermore, in various embodiments, the number of scores output by each of the prediction models 160A-160E can differ. For example, prediction model 160C shown in FIG. 1E can output one set of scores (hereafter referred to as “variant gene score” and “Order score”), and/or any one or more of prediction models 160A, 160B, 160D, and 160E shown in FIG. 1E can output respective sets of scores.

In each of the different embodiments of the prediction model described and shown in relation to FIGS. 1B-1E, each prediction model can be structured with sub-model architectures including one or more of: a binomial model and a multinomial model, as described in more detail below. Additionally or alternatively, sub-model architectures can include one or more of: a decision tree, an ensemble (e.g., bagging, boosting, random forest), gradient boosting machine, linear regression, Naïve Bayes, neural network, or logistic regression. Each prediction model includes learned coefficients for regression functions associated with different tissue sources of origin. Alternatively, in relation to different model architectures, prediction models or sub-models can include learned weights associated with training. The term weights is used generically here to represent the learned quantity associated with any given feature of a model, regardless of which particular machine learning technique is used.

During training, training data is processed to generate values for features that are used to train the coefficients and/or weights of the prediction model function(s). As an example, training data can include cfDNA and/or WBC DNA obtained from training samples, as well as an output label. For example, the label can indicate actual tissue source of origin related to presence of cancer in a subject from whom the training sample was sourced, can indicate whether the subject of the training sample is known to be cancerous or known to be devoid of cancer (e.g., healthy), and/or can indicate a severity of the cancer associated with the training sample. Depending on the particular embodiment shown in FIGS. 1B-1E, the prediction model receives the values for one or more of the features obtained from one or more of the physical assays and computational analyses relevant to the model to be trained. Depending on the differences between the scores output by the model-in-training and the output labels of the training data, the coefficients or weights of the functions of the prediction model are optimized enable the prediction model to make more accurate predictions.

The trained predictive cancer model can be stored and subsequently retrieved when needed, for example, during deployment in step 108 of FIG. 1A.

1.2. Physical Assays

FIG. 2A is flowchart of a method for performing a physical assay to prepare a nucleic acid sample for sequencing and to generate sequence reads, according to one embodiment that depicts step 104 of FIG. 1A in more detail. The method 104a includes, but is not limited to, the following steps. For example, any step of the method 104a can include a quantitation sub-step for quality control or other laboratory assay procedures known to one skilled in the art.

In step 210a, a test sample comprising a plurality of nucleic acid molecules (DNA or RNA) is obtained from a subject, and the nucleic acids are extracted and/or purified from the test sample. In the present disclosure, DNA and RNA can be used interchangeably unless otherwise indicated. That is, the following embodiments for using error source information in variant calling and quality control can be applicable to both DNA and RNA types of nucleic acid sequences. However, the examples described herein can focus on DNA for purposes of clarity and explanation. The nucleic acids in the extracted sample can comprise the whole human genome, or any subset of the human genome, including the whole exome. Alternatively, the sample can be any subset of the human transcriptome, including the whole transcriptome. The test sample can be obtained from a subject known to have or suspected of having cancer. In some embodiments, the test sample can include blood, plasma, serum, urine, fecal, saliva, other types of bodily fluids, or any combination thereof. Alternatively, the test sample can comprise a sample selected from the group consisting of whole blood, a blood fraction, a tissue biopsy, pleural fluid, pericardial fluid, cerebral spinal fluid, and peritoneal fluid. In some embodiments, methods for drawing a blood sample (e.g., syringe or finger prick) can be less invasive than procedures for obtaining a tissue biopsy, which can require surgery. The extracted sample can comprise cfDNA and/or ctDNA. For healthy individuals, the human body can naturally clear out cfDNA and other cellular debris. In general, any known method in the art can be used to extract and purify cell-free nucleic acids from the test sample. For example, cell-free nucleic acids can be extracted and purified using one or more known commercially available protocols or kits, such as the QIAamp circulating nucleic acid kit (QIAGEN®). If a subject has a cancer or disease, ctDNA in an extracted sample can be present at a detectable level for diagnosis.

In step 220a, a sequencing library is prepared. During library preparation, sequencing adapters comprising unique molecular identifiers (UMI) are added to the nucleic acid molecules (e.g., DNA molecules), for example, through adapter ligation (using T4 or T7 DNA ligase) or other known means in the art. The UMIs are short nucleic acid sequences (e.g., 4-10 base pairs) that are added to ends of DNA fragments and serve as unique tags that can be used to identify nucleic acids (or sequence reads) originating from a specific DNA fragment. Following adapter addition, the adapter-nucleic acid constructs are amplified, for example, using polymerase chain reaction (PCR). During PCR amplification, the UMIs are replicated along with the attached DNA fragment, which provides a way to identify sequence reads that came from the same original fragment in downstream analysis. Optionally, as is well known in the art, the sequencing adapters can further comprise a universal primer, a sample-specific barcode (for multiplexing) and/or one or more sequencing oligonucleotides for use in subsequent cluster generation and/or sequencing (e.g., known P5 and P7 sequences for used in sequencing by synthesis (SBS) (ILLUMINA®, San Diego, Calif.)).

In step 230a, targeted DNA sequences are enriched from the library. In accordance with some embodiments, during targeted enrichment, hybridization probes (also referred to herein as “probes”) are used to target, and pull down, nucleic acid fragments known to be, or that can be, informative for the presence or absence of cancer (or disease), cancer status, or a cancer classification (e.g., cancer type or tissue of origin). For a given workflow, the probes can be designed to anneal (or hybridize) to a target (complementary) strand of DNA or RNA. The target strand can be the “positive” strand (e.g., the strand transcribed into mRNA, and subsequently translated into a protein) or the complementary “negative” strand. The probes can range in length from 10 s, 100 s, or 1000 s of base pairs. In some embodiments, the probes are designed based on a gene panel to analyze particular mutations or target regions of the genome (e.g., of the human or another organism) that are suspected to correspond to certain cancers or other types of diseases. Moreover, the probes can cover overlapping portions of a target region. As one of skill in the art would readily appreciate, any known means in the art can be used for targeted enrichment. For example, the probes can be biotinylated and streptavidin coated magnetic beads used to enrich for probe captured target nucleic acids. See, e.g., Duncavage et al., J Mol Diagn. 13(3): 325-333 (2011); and Newman et al., Nat Med. 20(5): 548-554 (2014). By using a targeted gene panel rather than sequencing the whole genome (“whole genome sequencing”), all expressed genes of a genome (“whole exome sequencing” or “whole transcriptome sequencing”), the method 100 can be used to increase sequencing depth of the target regions, where depth refers to the count of the number of times a given target sequence within the sample has been sequenced. Increasing sequencing depth allows for detection of rare sequence variants in a sample and/or increases the throughput of the sequencing process. After a hybridization step, the hybridized nucleic acid fragments are captured and can also be amplified using PCR.

In step 240a, sequence reads are generated from the enriched nucleic acid molecules (e.g., DNA molecules). Sequencing data or sequence reads can be acquired from the enriched nucleic acid molecules by known means in the art. For example, the method 100 can include next generation sequencing (NGS) techniques including synthesis technology (ILLUMINA®), pyrosequencing (454 LIFE SCIENCES), ion semiconductor technology (Ion Torrent sequencing), single-molecule real-time sequencing (PACIFIC BIOSCIENCES®), sequencing by ligation (SOLiD sequencing), nanopore sequencing (OXFORD NANOPORE TECHNOLOGIES), or paired-end sequencing. In some embodiments, massively parallel sequencing is performed using sequencing-by-synthesis with reversible dye terminators.

In various embodiments, the enriched nucleic acid sample 215a is provided to the sequencer 245a for sequencing. As shown in FIG. 2A, the sequencer 245a can include a graphical user interface 250a that enables user interactions with particular tasks (e.g., initiate sequencing or terminate sequencing) as well as one more loading stations 155 for providing the sequencing cartridge including the enriched fragment samples and/or necessary buffers for performing the sequencing assays. Therefore, once a user has provided the necessary reagents and enriched fragment samples to the loading stations 255a of the sequencer 245a, the user can initiate sequencing by interacting with the graphical user interface 250a of the sequencer 245a. In step 240a, the sequencer 245a performs the sequencing and outputs the sequence reads of the enriched fragments from the nucleic acid sample 215.

In some embodiments, the sequencer 245a is communicatively coupled with one or more computing devices 260a. Each computing device 260a can process the sequence reads for various applications such as variant calling or quality control. The sequencer 245a can provide the sequence reads in a BAM file format to a computing device 260a. Each computing device 260a can be one of a personal computer (PC), a desktop computer, a laptop computer, a notebook, a tablet PC, or a mobile device. A computing device 260a can be communicatively coupled to the sequencer 245a through a wireless, wired, or a combination of wireless and wired communication technologies. Generally, the computing device 260a is configured with a processor and memory storing computer instructions that, when executed by the processor, cause the processor to process the sequence reads or to perform one or more steps of any of the methods or processes disclosed herein.

In some embodiments, the sequence reads can be aligned to a reference genome using known methods in the art to determine alignment position information. For example, in some embodiments, sequence reads are aligned to human reference genome hg19. The sequence of the human reference genome, hg19, is available from Genome Reference Consortium with a reference number, GRCh37/hg19, and also available from Genome Browser provided by Santa Cruz Genomics Institute. The alignment position information can indicate a beginning position and an end position of a region in the reference genome that corresponds to a beginning nucleotide base and end nucleotide base of a given sequence read. Alignment position information can also include sequence read length, which can be determined from the beginning position and end position. A region in the reference genome can be associated with a gene or a segment of a gene.

In various embodiments, for example when a paired-end sequencing process is used, a sequence read is comprised of a read pair denoted as R₁ and R₂. For example, the first read R₁ can be sequenced from a first end of a double-stranded DNA (dsDNA) molecule whereas the second read R₂ can be sequenced from the second end of the double-stranded DNA (dsDNA). Therefore, nucleotide base pairs of the first read R₁ and second read R₂ can be aligned consistently (e.g., in opposite orientations) with nucleotide bases of the reference genome. Alignment position information derived from the read pair R₁ and R₂ can include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format can be generated and output for further analysis such as variant calling.

FIG. 2B is flowchart of a method for performing a physical assay (e.g., a sequencing assay) to generate sequence reads, in accordance with another embodiment that depicts step 104 of FIG. 1A in more detail. The method 104b includes, but is not limited to, the following steps. For example, any step of the method 104b can comprise a quantitation sub-step for quality control or other laboratory assay procedures known to one skilled in the art.

Generally, various sub-combinations of the steps (e.g., steps 205b-235b) are performed for the small variant sequencing assay and/or one or more of: the whole genome sequencing assay, and methylation sequencing assay. For instance, Steps 205b and 215b-235b can be performed for the small variant sequencing assay. Additionally, in some embodiments, steps 205b, 215b, 230b, and 235b can be performed for the whole genome sequencing assay. Additionally, in some embodiments, each of steps 205b-235b are performed for the methylation sequencing assay. For example, a methylation sequencing assay that employs a targeted gene panel bisulfite sequencing employs each of steps 205b-235b. Alternatively, in some embodiments, steps 205b-215b and 230b-235b are performed for the methylation sequencing assay. For example, a methylation sequencing assay that employs whole genome bisulfite sequencing need not perform steps 220b and 225b.

At step 205b, nucleic acids (e.g., cfDNA) are extracted from a test sample, for instance, through a purification process. In general, any known method in the art can be used for purifying DNA. For example, nucleic acids can be isolated by pelleting and/or precipitating the nucleic acids in a tube. The extracted nucleic acids can include cfDNA or it can include gDNA, such as WBC DNA.

In step 210b, the cfDNA fragments are treated to convert unmethylated cytosines to uracils. In some embodiments, the method uses a bisulfite treatment of the DNA which converts the unmethylated cytosines to uracils without converting the methylated cytosines. For example, a commercial kit such as the EZ DNA METHYLATION—Gold, EZ DNA METHYLATION—Direct or an EZ DNA METHYLATION—Lightning kit (available from Zymo Research Corp, Irvine, Calif.) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.).

At step 215b, a sequencing library is prepared. During library preparation, adapters, for example, include one or more sequencing oligonucleotides for use in subsequent cluster generation and/or sequencing (e.g., known P5 and P7 sequences for use in sequencing by synthesis (SBS) (Illumina, San Diego, Calif.)) are ligated to the ends of the nucleic acid fragments through adapter ligation. In some embodiments, unique molecular identifiers (UMI) are added to the extracted nucleic acids during adapter ligation. The UMIs are short nucleic acid sequences (e.g., 4-10 base pairs) that are added to ends of nucleic acids during adapter ligation. In some embodiments, UMIs are degenerate base pairs that serve as a unique tag that can be used to identify sequence reads obtained from nucleic acids. As described later, the UMIs can be further replicated along with the attached nucleic acids during amplification, which provides a way to identify sequence reads that originate from the same original nucleic acid segment in downstream analysis.

In step 220b, hybridization probes are used to enrich a sequencing library for a selected set of nucleic acids. Hybridization probes can be designed to target and hybridize with targeted nucleic acid sequences to pull down and enrich targeted nucleic acid fragments that can be informative for the presence or absence of cancer (or disease), cancer status, or a cancer classification (e.g., cancer type or tissue of origin). In accordance with this step, a plurality of hybridization pull down probes can be used for a given target sequence or gene. The probes can range in length from about 40 to about 160 base pairs (bp), from about 60 to about 120 bp, or from about 70 bp to about 100 bp. In some embodiments, the probes cover overlapping portions of the target region or gene. In some embodiments, the hybridization probes are designed to enrich for DNA molecules that have been treated (e.g., using bisulfite) for conversion of unmethylated cytosines to uracils (i.e., the probes are designed to enrich for post-converted DNA molecules). In other embodiments, the hybridization probes are designed to enrich for DNA molecules that have not been treated (e.g., using bisulfite) for conversion of unmethylated cytosines to uracils (i.e., the probes are designed to enrich for pre-converted DNA molecules). For targeted gene panel sequencing, the hybridization probes are designed to target and pull down nucleic acid fragments that derive from specific gene sequences that are included in the targeted gene panel. For whole exome sequencing, the hybridization probes are designed to target and pull down nucleic acid fragments that derive from exon sequences in a reference genome.

After a hybridization step 220b, the hybridized nucleic acid fragments are enriched 225b. For example, the hybridized nucleic acid fragments can be captured and amplified using PCR. The target sequences can be enriched to obtain enriched sequences that can be subsequently sequenced. This improves the sequencing depth of sequence reads.

In step 230b, the nucleic acids are sequenced to generate sequence reads. Sequence reads can be acquired by known means in the art. For example, a number of techniques and platforms obtain sequence reads directly from millions of individual nucleic acid (e.g., DNA such as cfDNA or gDNA) molecules in parallel. Such techniques can be suitable for performing any of targeted gene panel sequencing, whole exome sequencing, whole genome sequencing, targeted gene panel bisulfite sequencing, and whole genome bisulfite sequencing.

As a first example, sequencing-by-synthesis technologies rely on the detection of fluorescent nucleotides as they are incorporated into a nascent strand of DNA that is complementary to the template being sequenced. In some methods, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle consists of adding the polymerase/labeled nucleotide mixture, rinsing, imaging and cleavage of dye.

In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain.

Any suitable sequencing-by-synthesis platform can be used to identify mutations. Sequencing-by-synthesis platforms include the Genome Sequencers from Roche/454 Life Sciences, the GENOME ANALYZER from Illumina/SOLEXA, the SOLID system from Applied BioSystems, and the HELISCOPE system from Helicos Biosciences. Sequencing-by-synthesis platforms have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that can dually serve as a universal primer.

As an alternative to a capture sequence, a member of a coupling pair (such as, e.g., antibody/antigen, receptor/ligand, or the avidin-biotin pair) can be linked to each fragment to be captured on a surface coated with a respective second member of that coupling pair. Subsequent to the capture, the sequence can be analyzed, for example, by single molecule detection/sequencing, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Massively parallel sequencing or next generation sequencing (NGS) techniques include synthesis technology, pyrosequencing, ion semiconductor technology, single-molecule real-time sequencing, sequencing by ligation, nanopore sequencing, or paired-end sequencing. Examples of massively parallel sequencing platforms are the Illumina HISEQ or MISEQ, ION PERSONAL GENOME MACHINE, the PACBIO RSII sequencer or SEQUEL System, Qiagen's GENEREADER, and the Oxford MINION. Additional similar current massively parallel sequencing technologies can be used, as well as future generations of these technologies.

At step 235b, the sequence reads can be aligned to a reference genome using known methods in the art to determine alignment position information. The alignment position information can indicate a beginning position and an end position of a region in the reference genome that corresponds to a beginning nucleotide base and end nucleotide base of a given sequence read. Alignment position information can also include sequence read length, which can be determined from the beginning position and end position. A region in the reference genome can be associated with a gene or a segment of a gene.

In various embodiments, a sequence read is comprised of a read pair denoted as R₁ and R₂. For example, the first read R₁ can be sequenced from a first end of a nucleic acid fragment whereas the second read R₂ can be sequenced from the second end of the nucleic acid fragment. Therefore, nucleotide base pairs of the first read R₁ and second read R₂ can be aligned consistently (e.g., in opposite orientations) with nucleotide bases of the reference genome. Alignment position information derived from the read pair R₁ and R₂ can include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary alignment map) format can be generated and output for further analysis.

Following step 235b, the aligned sequence reads are processed using a computational analysis, such as computational analysis 140B, 140C, or 140D as described above and shown in FIG. 1D. Each of the small variant computational analysis 140C, whole genome computation assay 140B, methylation computational analysis 140D, and baseline computational analysis are described in further detail below.

### 2. Small Variant Computational Analysis

2.1. Small Variant Features

The small variant computational analysis 140C described above in relation to FIGS. 1B-1E receives sequence reads generated by the small variant sequencing assay 134 and determines values of small variant features 154 based on the sequence reads, where the values of small variant features 154 can be assembled into a vector.

Examples of small variant features 154 include any of: a total number of somatic variants in a subject's cfDNA, a total number of nonsynonymous variants, total number of synonymous variants, a number of variants per gene represented in the sample, a presence/absence of somatic variants per gene in a gene panel, a presence/absence of somatic variants for particular genes that are known to be associated with cancer, an allele frequency (AF) of variants per gene in a gene panel, an AF of a somatic variant per category as designated by a publicly available database, such as oncoKB, another oncogenic-associated feature, a maximum variant allele frequency of a nonsynonymous variant associated with a gene, a ranked order of somatic variants according to the AF of somatic variants, other order statistics-associated features based on AF of somatic variants (e.g., a relative order statistics feature that represents a comparison of an allele frequency for a first variant to an allele frequency for at least one other variant), and/or features related to hotspot mutations, or mutation type such as nonsense or missense type mutations.

Additional examples of small variant features can include features describing one or more of: a classification of somatic variants that are known to be associated with cancer based on allele frequency, a mutation interaction describing joint presence of a first mutation and a second mutation for one or more genes (e.g., represented as a square root of a product of feature values corresponding to the first mutation and the second mutation). In relation to generation of predictions from processing the small variant features with a prediction model, the prediction model can preferentially return one candidate tissue source of origin over other candidate tissue sources of origin upon detection of one or a combination of features described above (or derived from features described above).

Generally, the feature values for the small variant features 154 are predicated on the accurate identification of somatic variants that can be indicative of a tissue source of origin related to cancer presence in a subject. The small variant computational analysis 140C identifies candidate variants and from amongst the candidate variants, differentiates between somatic variants likely present in the genome of the individual and false positive variants that are unlikely to be predictive of a tissue source of origin related to cancer presence in a subject. More specifically, the small variant computational analysis 140C identifies candidate variants present in cfDNA that are likely to be derived from a somatic source in view of interfering signals such as noise and/or variants that can be attributed to a genomic source (e.g., from gDNA or WBC DNA). Additionally candidate variants can be filtered to remove false positive variants that can arise due to an artifact and therefore are not indicative of cancer in the individual. As an example, false positive variants can be variants detected at or near the edge of sequence reads, which arise due to spontaneous cytosine deamination and end repair errors. Thus, somatic variants, and features thereof, that remain following the filtering out of false positive variants can be used to determine the small variant features.

For the feature of the total number of somatic variants, the small variant computational analysis 140C can total the identified somatic variants across the genome, or gene panel. Thus, for a cfDNA sample obtained from an individual, the feature of the total number of somatic variants can be represented as a single, numerical value of the total number of somatic variants identified in the cfDNA of the sample.

For the feature of the total number of nonsynonymous variants, the small variant computational analysis 140C can further filter the identified somatic variants to identify the somatic variants that are nonsynonymous variants. As is well known in the art, a non-synonymous variant of a nucleic acid sequence results in a change in the amino acid sequence of a protein associated with the nucleic acid sequence. For instance, non-synonymous variants can alter one or more phenotypes of an individual or cause (or leave more vulnerable) the individual to develop cancer, cancerous cells, or other types of diseases. Therefore, the small variant computation analysis 140C determines that a candidate variant would result in a non-synonymous variant by determining that a modification to one or more nucleobases of a trinucleotide would cause a different amino acid to be produced based on the modified trinucleotide. A feature value for the total number of nonsynonymous variants is determined by summating the identified nonsynonymous variants across the genome. Thus, for a cfDNA sample obtained from an individual, the feature of the total number of nonsynonymous variants can be represented as a single, numerical value.

For the feature of the total number of synonymous variants, synonymous variants represent other somatic variants that are not categorized as nonsynonymous variants. In other words, the small variant computational analysis 140C can perform the filtering of identified somatic variants, as described in relation to nonsynonymous variants, and identify the synonymous variants across the genome, or gene panel. Thus, for a cfDNA sample obtained from an individual, the feature of the total number of synonymous variants is represented as a single numerical value.

For feature of a presence/absence of somatic variants per gene can involve multiple feature values for a cfDNA sample. For example, a targeted gene panel can include 500 genes in the panel and therefore, the small variant computational analysis 140C can generate 500 feature values, each feature value representing either a presence or absence of somatic variants for a gene in the panel. As an example, if a somatic variant is present in the gene, then the value of the feature is 1. Conversely, if a somatic variant is not present in the gene, then the value of the feature is 0. In general, any size gene panel can be used. For example, the gene panel can comprise 100, 200, 500, 1000, 2000, 10,000 or more genes targets across the genome. some embodiments, the gene panel can comprise from about 50 to about 10,000 gene targets, from about 100 to about 2,000 gene targets, or from about 200 to about 1,000 gene targets.

For the feature of presence/absence of somatic variants for particular genes that are known to be associated with cancer, the particular genes known to be associated with cancer can be accessed from a public database such as OncoKB. Examples of genes known to be associated with cancer include TP53, LRP1B, and KRAS. Each gene known to be associated with cancer can be associated with a feature value, such as a 1 (indicating that a somatic variant is present in the gene) or a 0 (indicating that a somatic variant is not present in the gene).

The feature(s) representing the AF of a somatic variant per category can be determined by accessing a publicly available database, such as OncoKB. Chakravarty et al., JCO PO 2017. For example, OncoKB categorizes clinical information of genes in one of four different categories such as FDA approved, standard care, emerging clinical evidence, and biological evidence. Each such category can be its own feature having its own corresponding value. Other publicly available databases that can be accessed for determining features include the Catalogue Of Somatic Mutations In Cancer (COSMIC) and The Cancer Genome Atlas (TCGA) supported by the National Cancer Institutes' Genomic Data Commons (GDC). Forbes et al. COSMIC: somatic cancer genetics at high-resolution, Nucleic Acids Research, Volume 45, Issue D1, 4 Jan. 2017, Pages D777-D783. In some embodiments, the value of the AF of a somatic variant per category feature is determined as a maximum AF of a somatic variant across the genes in the category. In another embodiment, the value of the AF of a somatic variant per category feature is determined as a mean AF across somatic variants across the genes in the category. Measures other than max AF per category and mean AF per category can also be used.

The feature representing the AF of a somatic variant per gene (e.g., in a targeted gene panel) refers to a measure of the frequency of somatic variants in the sequence reads that relate to a particular gene. Generally, this feature is represented by one feature value per gene of a gene panel or per gene across the genome. The value of this feature can be a statistical value of AFs of somatic variants of the gene. The exact measurement used to prescribe a value to the feature can vary by embodiment. In some embodiments, the value for this feature is determined as the maximum AF of all somatic variants in the gene per position (e.g., in the genome). In some embodiments, the value for this feature is determined as the average AF of all somatic variants of the gene per position. Therefore, for an example targeted gene panel of 500 genes, there are 500 feature values that represent the AF of a somatic variant per gene. Measures other than max AF or mean AF can also be used.

The AF of a somatic variant per category can be determined according to categories as designated by a publicly available database, such as oncoKB. For example, oncoKB categorizes genes in one of four different categories. In some embodiments, the AF of a somatic variant per category is a maximum AF of a somatic variant across the genes in the category. In some embodiments, the AF of a somatic variant per category is a mean AF across somatic variants across the genes in the category.

The ranked order of somatic variants according to the AF of somatic variants refers to the top N allele frequencies of somatic variants. In general, the value of a variant allele frequency can be from 0 to 1, where a variant allele frequency of 0 indicates no sequence reads that possess the alternate allele at the position and where a variant allele frequency of 1 indicates that all sequence reads possess the alternate allele at the position. In other embodiments, other ranges and/or values of variant allele frequencies can be used. In various embodiments, the ranked order feature is independent of the somatic variants themselves and instead, is only represented by the values of the top N variant allele frequencies. An example of the ranked order feature for the top 5 allele frequencies can be represented as: [0.1, 0.08, 0.05, 0.03, 0.02] which indicates that the 5 highest allele frequencies, independent of the somatic variants, range from 0.02 up to 0.1.

2.2. Small Variant Computational Analysis Process Overview

A processing system, such as a processor of a computer, executes the code for performing the small variant computational analysis 140C.

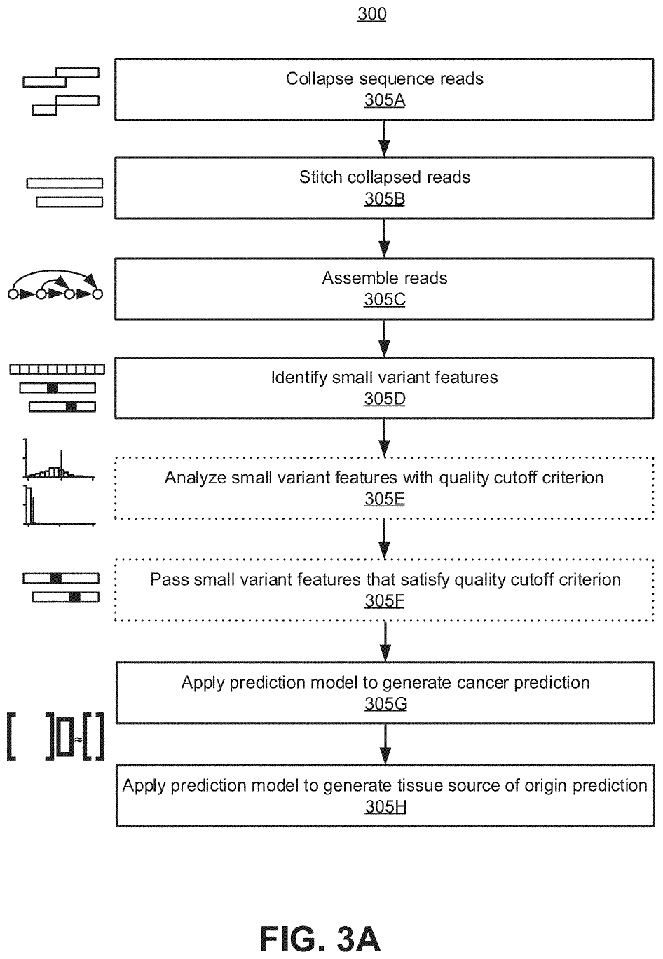

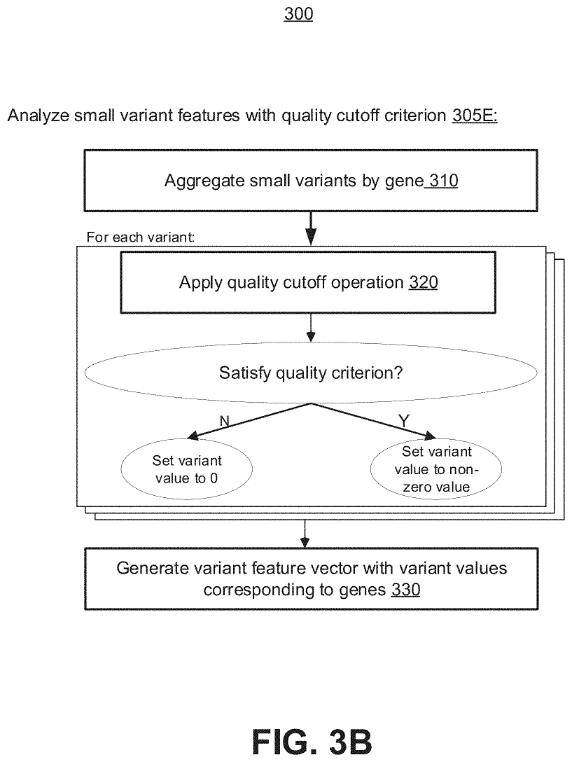

FIG. 3A is flowchart of a method 300 for determining somatic variants from sequence reads, in accordance with some embodiments. At step 305A, the processing system collapses aligned sequence reads. In some examples, collapsing sequence reads includes using UMIs, and optionally alignment position information from sequencing data of an output file to collapse multiple sequence reads into a consensus sequence for determining the most likely sequence of a nucleic acid fragment or a portion thereof. The unique sequence tag can be from about 4 to 20 nucleic acids in length. Since the UMIs are replicated with the ligated nucleic acid fragments through enrichment and PCR, the sequence processor 205 can determine that certain sequence reads originated from the same molecule in a nucleic acid sample. In some embodiments, sequence reads that have the same or similar alignment position information (e.g., beginning and end positions within a threshold offset) and include a common UMI are collapsed, and the processing system generates a collapsed read (also referred to herein as a consensus read) to represent the nucleic acid fragment. The processing system designates a consensus read as “duplex” if the corresponding pair of collapsed reads have a common UMI, which indicates that both positive and negative strands of the originating nucleic acid molecule is captured; otherwise, the collapsed read is designated “non-duplex.” In some embodiments, the processing system can perform other types of error correction on sequence reads as an alternative to, or in addition to, collapsing sequence reads.

At step 305B, the processing system stitches the collapsed reads based on the corresponding alignment position information. In some embodiments, the processing system compares alignment position information between a first sequence read and a second sequence read to determine whether nucleotide base pairs of the first and second sequence reads overlap in the reference genome. In one use case, responsive to determining that an overlap (e.g., of a given number of nucleotide bases) between the first and second sequence reads is greater than a threshold length (e.g., threshold number of nucleotide bases), the processing system designates the first and second sequence reads as “stitched”; otherwise, the collapsed reads are designated “unstitched.” In some embodiments, a first and second sequence read are stitched if the overlap is greater than the threshold length and if the overlap is not a sliding overlap. For example, a sliding overlap can include a homopolymer run (e.g., a single repeating nucleotide base), a dinucleotide run (e.g., two-nucleotide base sequence), or a trinucleotide run (e.g., three-nucleotide base sequence), where the homopolymer run, dinucleotide run, or trinucleotide run has at least a threshold length of base pairs.

At step 305C, the processing system assembles reads into paths. In some embodiments, the processing system assembles reads to generate a directed graph, for example, a de Bruijn graph, for a target region (e.g., a gene). Unidirectional edges of the directed graph represent sequences of k nucleotide bases (also referred to herein as “k-mers”) in the target region, and the edges are connected by vertices (or nodes). The processing system aligns collapsed reads to a directed graph such that any of the collapsed reads can be represented in order by a subset of the edges and corresponding vertices.

In some embodiments, the processing system determines sets of parameters describing directed graphs and processes directed graphs. Additionally, the set of parameters can include a count of successfully aligned k-mers from collapsed reads to a k-mer represented by a node or edge in the directed graph. The processing system stores directed graphs and corresponding sets of parameters, which can be retrieved to update graphs or generate new graphs. For instance, the processing system can generate a compressed version of a directed graph (e.g., or modify an existing graph) based on the set of parameters. In some example use cases, in order to filter out data of a directed graph having lower levels of importance, the processing system removes (e.g., “trims” or “prunes”) nodes or edges having a count less than a threshold value, and maintains nodes or edges having counts greater than or equal to the threshold value.

At step 305D, the processing system identifies candidate small variant features from the assembled reads. In some embodiments, the processing system identifies candidate small variant features by comparing a directed graph (which may have been compressed by pruning edges or nodes in step 305B) to a reference sequence of a target region of a genome. The processing system can align edges of the directed graph to the reference sequence, and records the genomic positions of mismatched edges and mismatched nucleotide bases adjacent to the edges as the locations of candidate small variants. In some embodiments, the genomic positions of mismatched edges and mismatched nucleotide bases to the left and right of edges are recorded as the locations of called variants. Additionally, the processing system can generate candidate small variants based on the sequencing depth of a target region. In particular, the processing system can be more confident in identifying variants in target regions that have greater sequencing depth, for example, because a greater number of sequence reads help to resolve (e.g., using redundancies) mismatches or other base pair variations between sequences.

In some embodiments, the processing system identifies candidate small variant features using a model to determine expected noise rates for sequence reads from a subject. The model can be a Bayesian hierarchical model, though in some embodiments, the processing system uses one or more different types of models. Moreover, a Bayesian hierarchical model can be one of many possible model architectures that can be used to generate candidate variants and which are related to each other in that they all model position-specific noise information in order to improve the sensitivity/specificity of variant calling. More specifically, the processing system trains the model using samples from healthy individuals to model the expected noise rates per position of sequence reads.

Further, multiple different models can be stored in a database or retrieved for application post-training. For example, a first model is trained to model SNV noise rates and a second model is trained to model insertion deletion noise rates. Further, the processing system can use parameters of the model to determine a likelihood of one or more true positives in a sequence read. The processing system can determine a quality score (e.g., on a logarithmic scale) based on the likelihood. For example, the quality score is a Phred quality score Q=−10·log₁₀ P, where P is the likelihood of an incorrect candidate variant call (e.g., a false positive). Other models, such as a joint model, can use output of one or more Bayesian hierarchical models to determine expected noise of nucleotide mutations in sequence reads of different samples (e.g., per position).

At step 305E, the processing system analyzes the small variant features with a quality cutoff criterion, and in step 305F, passes small variant features that satisfy the quality cutoff criterion, where embodiments of a quality cutoff criterion operation are described in relation to FIG. 3B. In step 305G, the processing system applies the prediction model (e.g., an embodiment of the prediction model described in relation to FIGS. 1A-1E above) to generate a prediction indicating cancer presence or absence and in step 305H, the processing system applies the prediction model (e.g., an embodiment of the prediction model described in relation to FIGS. 1A-1E above) to generate a prediction of tissue source of origin related to cancer presence in the subject. FIG. 3B depicts a flowchart of step 305E shown in FIG. 3A for applying a quality cutoff criterion to candidate small variant features, in accordance with an embodiment. At step 310, the processing system aggregates small variants by gene. Then, for each variant, the processing system applies a quality cutoff criterion in step 320 where, if the quality criterion is satisfied, the value of the small variant feature is set to a non-zero value (as described above in relation to small variant feature values). In some embodiments, if the quality criterion is satisfied, the value of the small variant feature is set to the maximum allele frequency (max(AF)). Conversely, if the quality criterion is not satisfied, the processing system sets the value of the small variant feature to zero. Then, in step 330A, the processing system generates a variant feature vector with variant values corresponding to respective genes. In some variations, depending on the level of satisfaction of the quality criterion, a weight can be applied to the value of the small variant feature, where, for example, a small variant feature that satisfies the quality criterion to a high degree has a more heavily weighted value. Furthermore, in some embodiments, the quality cutoff criterion is only applied to coding regions of a sequence; however, the quality cutoff criterion can additionally or alternatively be applied to non-coding regions of a sequence.

In various embodiments, generating candidate variants and/or performing computational analyses in a joint model for processing outputs of sequencing assays can be implemented according to embodiments described in U.S. application Ser. No. 16/201,912 titled “Models for Targeted Sequencing” and filed on 27 Nov. 2018, now published as U.S. App. Pub. No. 2019/0164627, which is herein incorporated in its entirety.

Furthermore, as described above, outputs of the computational analyses for processing outputs of a small variant sequencing assay can be used by the processing system to derive relevant copy number features. In embodiments, a set of copy number features can include a focal copy number of a mutation, the focal copy number describing repetition of a genetic variation represented in below a threshold proportion of a sequence from a cfDNA sample. The set of copy number features can additionally or alternatively include a copy number feature associated with a fusion or a structural variant.

### 3. Computational Analysis of Other Features

Computational analyses of other features can be performed according to embodiments described in U.S. App. No. 62/657,635 titled “Multi-Assay Prediction Model for Cancer Detection” and filed on 13 Apr. 2018, now included by priority claim in U.S. App. Pub. No. 2019/0316209, filed on 15 Apr. 2019 and titled “Multi-Assay Prediction Model for Cancer Detection,” and according to embodiments described in U.S. application Ser. No. 16/417,336, filed on 20 May 2019 and titled “Inferring Selection in White Blood Cell Matched Cell-free DNA Variants and/or in RNA Variants,” the contents of all which are herein incorporated in their entirety.

### 4. Prediction Model Architecture

4.1. First Sub-Model

In relation to different sub-models of the prediction model used to generate a cancer prediction (described above in relation to FIG. 3A, step 305G), the first sub-model can be structured as a binary classification model (e.g., as part of an elastic-net classification package) that outputs a prediction, with or without an associated confidence, identifying the sample as cancerous or non-cancerous. The binary classification can allow for a non-negative coefficient output where the magnitude of the coefficient corresponds to increased likelihood of classification to a cancerous condition. In some cases, the binary classification is restricted to non-negative coefficient outputs. Still, in some examples, the binary classification can also allow for a negative coefficient output corresponding to decreased likelihood of classification to a cancerous condition. However, in alternative variations, the binary classification can output a coefficient having a coefficient direction and/or magnitude corresponding to a cancerous or non-cancerous condition in any other suitable manner.

Furthermore, the binary classification model can include an alpha parameter configured to tune performance of the first sub-model between a ridge-like regression mode and a lasso-like regression mode, where the method can implement architecture for evaluating a contribution of each of the set of small variant features to the prediction and adjusting the alpha parameter based upon the contributions. In relation to the alpha parameter, adjustment of alpha for the ridge-like regression mode can, in relation to model behavior, punish high values of the coefficients of the binomial classification model by reducing the magnitudes of such coefficients, thereby minimizing their impact on the trained models. In relation to the alpha parameter, adjustment of alpha for the lasso-like regression mode can, in relation to model behavior, punish high values of the coefficients of the binomial classification model by setting high values of non-relevant coefficients to zero. As such, the binary classification model can be a penalized binomial classification model that can be tuned, by the alpha parameter, for inclusion of features strongly classifying samples as cancerous or non-cancerous.

In relation to a prediction score output of the binary classification architecture of the first sub-model, the prediction score can be generated based on processing a set of features (e.g., small variant features) as input features, where the set of features are associated with cancer presence or non-presence. The prediction score can then be compared to a threshold condition, where satisfaction of the threshold condition indicates cancer presence and non-satisfaction of the threshold condition indicates cancer non-presence.

The binary classification model can also include a specificity condition characterizing cancer signal strength, where the specificity condition provides an initial filter for samples from individuals with a highly-specific cancer signal. The specificity condition can be a threshold specificity (e.g., of 99.9% specificity, of 99% specificity, of 98% specificity, of 95% specificity, etc.), where, if the specific condition is satisfied by the output of the binary classification model, the sample is processed with the second sub-model of the prediction model (e.g., a multinomial model, as described below). In some examples, the binomial threshold specificity is selected based on the non-cancer population (e.g., selected from a distribution of prediction scores predicted by the binary classification model for non-cancer samples), and any sample having a score above the score corresponding to the threshold specificity is examined further with the multinomial classification model.

The binary classification model can, however, be constructed with other filters or conditions (e.g., sensitivity condition, non-specificity conditions, non-sensitivity conditions) for generation of derivative outputs of the prediction model at different stages. Furthermore, the first sub-model can have another architecture (e.g., random forest model architecture, gradient boosting machine architecture, etc.).

4.2. Second Sub-Model

In relation to different sub-models of the prediction model, the second sub-model can be structured as a multinomial classification model (e.g., as part of an elastic-net classification package) that outputs a prediction, with or without an associated confidence, identifying the tissue source of origin for the cancer as belonging to one or more of a set of candidate tissue sources. The multinomial classification model can be a multinomial regression model that outputs a set of values, each value indicating a probability that the cancer associated with the sample originated from one of the set of candidate tissue sources associated with that value.

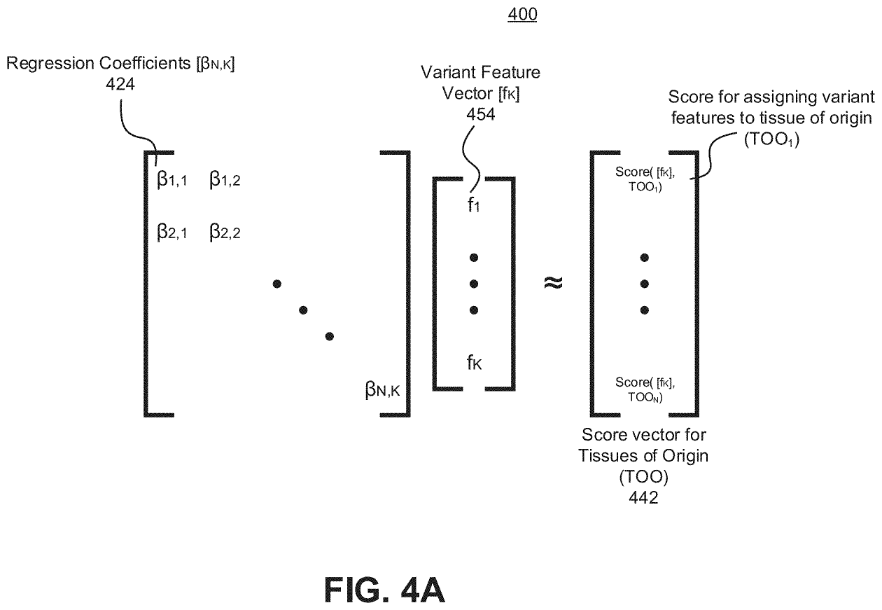

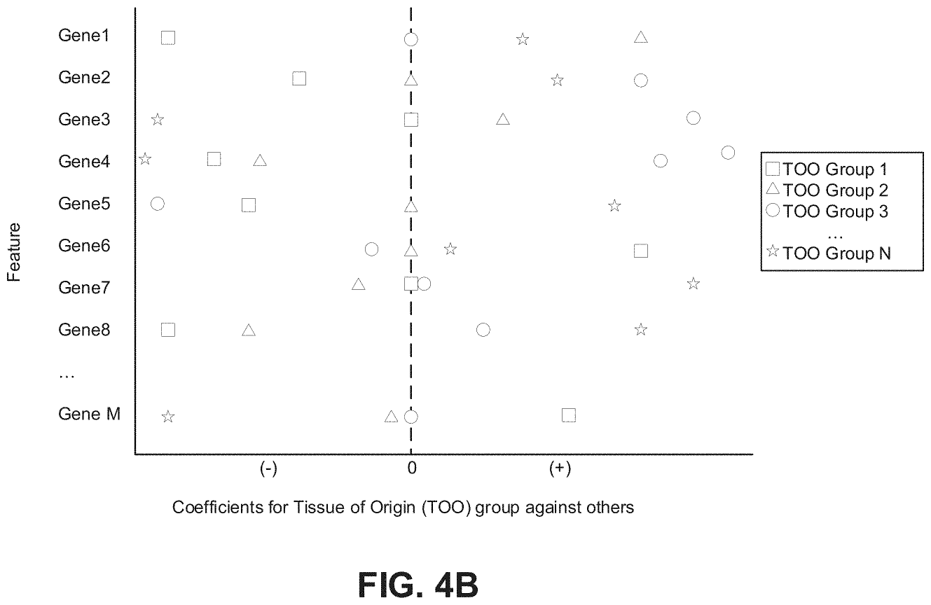

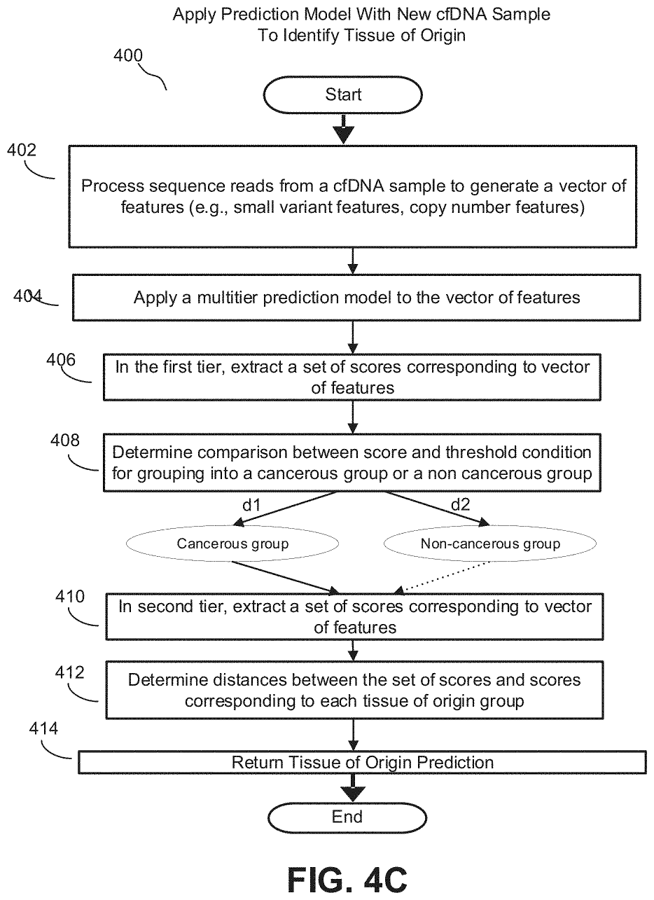

FIG. 4A depicts an example of a model architecture for processing a feature vector (e.g., a feature vector of small variant features) to predict tissue source of origin. In the example shown in FIG. 4A, the set of features, arranged as a vector, is processed with a penalized multinomial regression model. In the example shown in FIG. 4A, the penalized multinomial regression model is arranged as a set of regressions, where, a matrix of regression coefficients (β_{1,1} through β_{N,K}), applied to a variant feature vector containing values (f₁ through f_{K}) of proposed explanatory features (e.g., small variant features corresponding to different genes of interest) produces a vector of scores (Score ([f], TOO₁) through Score ([f], TOO_{N}) for assigning features to a tissue source of origin group. In the example shown in FIG. 4A, there are N possible tissue source of origin groupings, and K features of interest. Generally, the model can be constructed as Score=β*f, where the score can indicate the probability of a sample belonging to a particular tissue source of origin group, based on features observed through processing of the sample.

In determining the coefficients through training of the penalized multinomial regression model, the processing system can run, for N possible groupings (corresponding to tissue sources of origin), N−1 binary regression models where, for each binary regression model one tissue source of origin group serves as a “pivot” and the remaining N−1 tissue source of origin groups are separately regressed against the “pivot”. In more detail, for a specific example of one binary regression of the multinomial regression, a breast tissue source of origin can serve as a “pivot” against which the other tissue sources of origin (e.g., colorectal, head and neck, ovarian, etc.) are regressed. Then, the scores (or probabilities) associated with each regression are determined based on the condition that all probabilities must add to one. In solving the probabilities, the coefficients of β are estimated (e.g., using a maximum a posteriori (MAP) estimation, using a maximum likelihood approach, using another approach). Determination of the scores and estimated coefficients corresponding to small variant (or other) features for each tissue source of origin grouping is performed across a training dataset where the tissue sources of origin associated with training samples is known.

The penalized multinomial regression model thus defines a set of functions with a set of coefficients trained by a dataset, where the training dataset can be derived from cfDNA samples of a population of subjects. The functions can be logistic functions or other functions. The multinomial regression model can be trained with at least eight cfDNA samples for each of a set of candidate of tissue sources; however, the multinomial regression model can alternatively be trained with any other suitable number of training samples. In some examples, samples known to have multiple cancers (e.g., more than one cancer type) are removed to restrict the training dataset down to the samples where tissue of origin can be reasonably trained. Further, in some examples, training datasets can also include training data from tissue samples (i.e., gDNA).

Similar to the description of the binary classification model architecture, the multinomial regression model can include an alpha parameter configured to tune performance of the second sub-model between a ridge-like regression mode and a lasso-like regression mode, where the method can implement architecture for evaluating a contribution of each of the set of small variant features to the prediction and adjusting the alpha parameter based upon the contributions. In relation to the alpha parameter, adjustment of alpha for the ridge-like regression mode can, in relation to model behavior, punish high values of the coefficients of the multinomial regression model by reducing the magnitudes of such coefficients, thereby minimizing their impact on the trained models. In relation to the alpha parameter, adjustment of alpha for the lasso-like regression mode can, in relation to model behavior, punish high values of the coefficients of the multinomial regression model by setting high values of non-relevant coefficients to zero. As such, the multinomial regression model can be a penalized multinomial regression model that can be tuned, by the alpha parameter, for inclusion of features strongly classifying samples as to different tissue source of origin groups.

The multinomial regression model can also include a specificity condition that characterizes performance of the multinomial regression model. The specificity condition can be a threshold specificity (e.g., of 99.9% specificity, of 99% specificity, of 98% specificity, of 95% specificity, etc.). The multinomial regression model can also include a sensitivity condition that characterizes performance of the multinomial regression model. The sensitivity condition can be a threshold sensitivity (e.g., of 40% sensitivity, of 50% sensitivity, of 60% sensitivity, of 70% sensitivity, etc.). Furthermore, performance of the prediction model can be evaluated by different specificity conditions and/or sensitivity conditions, based on application of the prediction model. For instance, specificity conditions and/or sensitivity conditions can vary when using the model for screening, as opposed to using the model for evaluating higher risk and/or higher frequency populations of subjects. In some examples, performance of the predictive model is characterized by at least a 50% sensitivity at a 99% specificity when applying the predictive model for screening purposes. In other examples, performance of the predictive model is characterized by at least a 60% sensitivity at a 95% specificity when applying the predictive model for higher risk and higher frequency populations. In some examples, the specificity and/or sensitivity of the multiclass and/or binary classifier can be user set or otherwise adjustable by the user.

The multinomial model can, however, be constructed with other filters or conditions (e.g., sensitivity condition, non-specificity conditions, non-sensitivity conditions) for evaluating model performance. Furthermore, the second sub-model can have another architecture. For instance, the second sub-model can include a support vector machine with architecture for evaluating each of the set of candidate tissue sources against other candidate tissue sources of the set of candidate tissue sources. Alternatively, the second sub-model can include a random forest classifier with learned weights derived from samples from a population of subjects. Alternatively, the second sub-model can include a gradient boosting machine.

FIG. 4B depicts an embodiment of model coefficient outputs for features associated with different genes, in relation to predictions of tissue sources of origin. In FIG. 4B, features corresponding to a set of genes (Gene1 through Gene M) are depicted along the y-axis, and regression model coefficients are represented on the x-axis. As shown in FIG. 4B, for each of a set of tissue source of origin groups, the trained prediction model can include, for each of a set of features corresponding to a set of relevant genes (e.g., Gene1 through Gene M), a set of coefficients corresponding to a regression of the set of features for the tissue source of origin (i.e., the pivot) against other tissue sources of origin. As shown in FIG. 4B, for tissue source of origin group 1 (TOO Group 1), the model includes coefficient values for each feature associated with Gene1 through Gene M (represented as squares in the graph). Similarly, for tissue source of origin group 2 (TOO Group 2), the model includes coefficient values for each feature associated with Gene1 through Gene M (represented as triangles in the graph). Similarly, for tissue source of origin group 3 (TOO Group 3), the model includes coefficient values for each feature associated with Gene1 through Gene M (represented as circles in the graph). Similarly, for tissue source of origin group N (TOO Group N), the model includes coefficient values for each feature associated with Gene1 through Gene M (represented as stars in the graph). For each coefficient, the magnitude and the direction (e.g., positive or negative direction) are indicative of likelihood of a coefficient being relevant. In more detail, and as shown in FIG. 4B, the prediction model can allow for a negative coefficient output corresponding to decreased likelihood of classification to a first tissue source of the set of tissue sources of origin (e.g., as for TOO Group 1 and feature for Gene1 in FIG. 4B), a zero coefficient output corresponding to indeterminate classification (e.g., as for TOO Group 2 and feature for Gene6 in FIG. 4B), and a positive coefficient output corresponding to increased likelihood of classification to the first tissue source of the set of candidate tissue sources (e.g., as for TOO Group 3 and feature for Gene2 in FIG. 4B). In relation to coefficient magnitudes and directions, during determination of the coefficient values of the prediction model, the coefficient magnitudes can be reduced or set to zero, according to a penalization process, depending on feature relevance to generation of a prediction, as indicated above in relation to the alpha parameter(s).

4.3. Prediction Model Application

FIG. 4C depicts a flow process for applying an embodiment of a prediction model to a feature vector derived from a sample from a subject, to return a tissue source of origin prediction, in accordance with some embodiments. For a non-training sample, FIG. 4C depicts a process 400 for processing the sample to extract features of interest, and then applying a prediction model, such as an embodiment of a prediction model described above, to features extracted from the sample in order to generate a tissue source of origin prediction associated with cancer presence (described above in relation to FIG. 3A, steps 305G and/or 305H). In more detail, as shown in FIG. 4C, in Step 402, a processing system (such as the processing system described above in relation to FIG. 3A) processes sequence reads from a cfDNA sample from a subject to generate a vector of features (e.g., small variant features, copy number features, etc., as described above in relation to FIG. 3A, steps 305A-305G). Processing the cfDNA sample can be performed as described above.

Then, in Step 404, the processing system applies the prediction model (e.g., a first sub-model for generating a cancerous vs. non-cancerous prediction and a second sub-model for generating a tissue source of origin prediction). In more detail, in Step 406, the processing system extracts a score upon processing the set of features from the cfDNA sample with a trained first sub-model of the prediction model. Then, the processing system, in Step 408, compares the score determined for the sample and a threshold condition corresponding to a cancerous grouping vs. a non-cancerous grouping. If the score for the cfDNA sample satisfies the threshold condition associated with a cancerous grouping, the prediction model outputs a prediction associating the sample with a cancerous grouping. Conversely, if the score for the cfDNA sample does not satisfy the threshold condition for a cancerous grouping, the prediction model outputs a prediction associating the sample with a non-cancerous grouping.

In Step 410, the processing system extracts a set of coefficients upon processing a set of features from the cfDNA sample (where the set of features can be the same features or features different from features processed with the first sub-model described above) and compares the set of coefficients with coefficients of a trained second sub-model of the prediction model. Then, the processing system, in Step 408 determines distances between the coefficients determined for the sample and sets of coefficients corresponding to each of a set of tissue sources of origin groupings. Sets of coefficients corresponding to the sample and sets of coefficients corresponding to each of the set of tissue sources of origin can be arranged as vectors, where distances between vectors can be determined according to Euclidean distance calculations or another suitable method. If the distance between the coefficients for the cfDNA sample and that for particular tissue source of origin is smaller than the distance between the coefficients for the cfDNA sample and that for other tissue sources of origin groupings, the prediction model outputs a prediction associating the sample with the particular tissue source of origin corresponding to the minimum distance in scores.

In relation to coefficient magnitudes and directions, the prediction model can generate predictions based on a value of a single feature or values of multiple features. For instance, the prediction model can include a positive coefficient (e.g., a positive coefficient with a high magnitude different than that for other tissue sources of origin) corresponding to a feature of the set of features (e.g., a small variant feature of a particular gene), and processing the set of features to generate a tissue source of origin prediction from the cfDNA sample can include: identifying, from the cfDNA sample, a signal corresponding to the feature associated with the positive coefficient, and outputting, from the prediction model, a candidate tissue source of the set of candidate tissue sources as the prediction based on presence of the feature in association with the cfDNA sample.

In another example, the prediction model can include a negative coefficient (e.g., a negative coefficient with a high magnitude different than that for other tissue sources of origin) corresponding to a feature of the set of features (e.g., a small variant feature of a particular gene), and processing the set of features to generate a tissue source of origin prediction from the cfDNA sample can include: identifying, from the cfDNA sample, a signal corresponding to the feature associated with the negative coefficient, and excluding a candidate tissue source of the set of candidate tissue sources from the prediction based on presence of the feature in association with the cfDNA sample.

### 5. Example Prediction Model Coefficients for Different Tissue Sources of Origin

The example model coefficients shown below in TABLES 3-23 were determined through training of a multinomial regression model using a training data set obtained from training samples. As shown in TABLE 1, the training samples (N=1453) were blood samples collected from individuals diagnosed with cancer (N=879) and healthy individuals with no cancer diagnosis (N=574). Cell-free DNA were extracted from the samples, sequenced, and analyzed for features (e.g., non-synonymous informative variants within a gene) to produce training data for the training data set. A breakdown of the cancer samples (N=879) by cancer type is provided in TABLE 2. The final training data set was filtered to remove some samples based on quality control thresholds or issues, such as discovery of an unreliable flow cell that was included in the data set.

TABLE 1
Samples used for training.

| | N |
|---|---|
| Cancer | 879 |
| Non-cancer | 574 |
| Total samples | 1453 |

TABLE 2
Cancer samples by cancer type.

| | N |
|---|---|
| Bladder | 11 |
| Breast | 357 |
| Cancer of unknown primary | 0 |
| Cervical | 13 |
| Colorectal | 50 |
| Esophageal | 25 |
| Gastric | 12 |
| Head/Neck | 20 |
| Hepatobiliary | 15 |
| Leukemia | 13 |
| Lung | 125 |
| Lymphoma | 25 |
| Melanoma | 11 |
| Multiple myeloma | 14 |
| Other | 0 |
| Ovarian | 21 |
| Pancreas | 27 |
| Prostate | 71 |
| Renal | 28 |
| Thyroid | 13 |
| Uterine | 28 |

5.1. Example Bladder Tissue Source of Origin Coefficients

TABLE 3 provides an example of model coefficient outputs for features associated with different genes, in relation to a prediction of a bladder tissue source of origin, where model coefficients were determined from a sample data set and a training data set from at least 8 cfDNA samples. As shown in TABLE 3, a multinomial regression model can have coefficients corresponding to small variant features for different genes, in a regression between the small variant features and bladder tissue against other tissue groups. Representative coefficient values, corresponding to small variant features for a set of genes (e.g., top 14 ranked features based on absolute value), are shown in TABLE 3, where positive coefficient values indicate evidence for a bladder tissue source, in relation to tissue source of origin, and negative coefficient values indicate evidence for another type of cancer, in relation to tissue source of origin.

TABLE 3
Coefficients for Gene Variant Features related
to a Bladder Tissue Source of Origin

| Feature | Coefficient Value |
|---|---|
| TSC1 | 16 |
| TP53 | 1 |
| TNFRSF14 | 9.5 |
| RANBP2 | 30 |
| MTOR | 23 |
| MSH6 | 28 |
| KRAS | −7 |
| KDM6A | 33.5 |
| JAK2 | 64 |
| ESR1 | 4 |
| ERBB2 | 5 |
| CBL | 4 |
| BRCA1 | 11 |
| BAP1 | 7 |

As such, in relation to outputting a prediction according to embodiments of method steps described above, the processing system can generate a prediction of bladder tissue as the tissue source of origin upon evaluating values of the set of features corresponding to one or more, two or more, three or more, four or more, five or more, eight or more, or ten or more, of a set of small variant features listed in TABLE 3. In some examples, a gene panel (e.g., targeted sequencing panel for generating a prediction of bladder tissue source of origin) can include genes and/or gene features corresponding to the one or more, two or more, three or more, four or more, five or more, eight or more, or ten or more gene features listed in TABLE 3.

5.2. Example Breast Tissue Source of Origin Coefficients

An example of model coefficient outputs for features associated with different genes, in relation to a prediction of a breast tissue source of origin, and representative coefficient values, corresponding to small variant features for a set of genes (e.g., top 14 ranked features based on absolute value), are shown in TABLE 4. For example, as shown in TABLE 4, features related to PIK3CA variants provide positive evidence for a breast cancer type, while features related to LRP1B variants provide negative evidence (i.e., that the tissue source of origin is probably not breast but rather another cancer type), and further that presence of features related to KRAS variants provide strong negative evidence (e.g., extreme negative coefficient) that the tissue source of origin is most likely not breast.

TABLE 4
Coefficients for Gene Variant Features related
to a Breast Tissue Source of Origin

| Feature | Coefficient Value |
|---|---|
| TP53 | 35 |
| TNFRSF14 | −30 |
| SLIT2 | −41 |
| PTPRT | −35 |
| PTCH1 | 40 |
| PIK3CA | 49.5 |
| LRP1B | −57 |
| KRAS | −91 |
| GATA3 | 40 |
| FLT1 | 33 |
| FBXW7 | 34 |
| FANCD2 | 34 |
| ERBB4 | −33 |
| BRAF | −37.5 |

As such, in relation to outputting a prediction according to embodiments of method steps described above, the processing system can generate a prediction of breast tissue as the tissue source of origin upon evaluating values of the set of features corresponding to one or more, two or more, three or more, four or more, five or more, eight or more, or ten or more, of a set of small variant features listed in TABLE 4. In some examples, a gene panel (e.g., targeted sequencing panel for generating a prediction of breast tissue source of origin) can include genes and/or gene features corresponding to the one or more, two or more, three or more, four or more, five or more, eight or more, or ten or more gene features listed in TABLE 4.

5.3. Example Cervical Tissue Source of Origin Coefficients

An example of model coefficient outputs for features associated with different genes, in relation to a prediction of a cervical tissue source of origin, and representative coefficient values, corresponding to small variant features for a set of genes (e.g., top 14 ranked features), are shown in TABLE 5.

TABLE 5
Coefficients for Gene Variant Features related
to a Cervical Source Tissue of Origin

| Feature | Coefficient Value |
|---|---|
| TP63 | 12 |
| TP53 | −16 |
| RFWD2 | 29 |
| PIK3CA | 17 |
| KRAS | −4 |
| KMT2C | 10 |
| KIT | 4 |
| DICER1 | 6 |
| CHD2 | 7 |
| CCND3 | 76 |
| BLM | 13 |
| ATM | 13.5 |
| ARID1A | 12 |
| AKT3 | 14 |

As such, in relation to outputting a prediction according to embodiments of method steps described above, the processing system can generate a prediction of cervical tissue as the tissue source of origin upon evaluating values of the set of features corresponding to one or more, two or more, three or more, four or more, five or more, eight or more, or ten or more, of a set of small variant features listed in TABLE 5. In some examples, a gene panel (e.g., targeted sequencing panel for generating a prediction of cervix tissue source of origin) can include genes and/or gene features corresponding to the one or more, two or more, three or more, four or more, five or more, eight or more, or ten or more gene features listed in TABLE 5.

5.4. Example Colorectal Tissue Source of Origin Coefficients

An example of model coefficient outputs for features associated with different genes, in relation to a prediction of a colorectal tissue source of origin, and representative coefficient values, corresponding to small variant features for a set of genes (e.g., top 14 ranked features), are shown in TABLE 6.

TABLE 6
Coefficients for Gene Variant Features related
to a Colorectal Source Tissue of Origin

| Feature | Coefficient Value |
|---|---|
| SPEN | 33 |
| RUNX1T1 | 27 |
| PTEN | 75 |
| PIK3CA | 51 |
| PAX3 | 25 |
| LRP1B | 37.5 |
| KRAS | 85 |
| KLF4 | 35 |
| KIF5B | 42 |
| JAK2 | 25 |
| ESR1 | 31 |
| BRAF | 37 |
| APC | 95 |
| AMER1 | −24 |

As such, in relation to outputting a prediction according to embodiments of method steps described above, the processing system can generate a prediction of colorectal tissue as the tissue source of origin upon evaluating values of the set of features corresponding to one or more, two or more, three or more, four or more, five or more, eight or more, or ten or more, of a set of small variant features listed in TABLE 6. In some examples, a gene panel (e.g., targeted sequencing panel for generating a prediction of colorectal tissue source of origin) can include genes and/or gene features corresponding to the one or more, two or more, three or more, four or more, five or more, eight or more, or ten or more gene features listed in TABLE 6.

5.5. Example Esophageal Tissue Source of Origin Coefficients

An example of model coefficient outputs for features associated with different genes, in relation to a prediction of an esophageal tissue source of origin, and representative coefficient values, corresponding to small variant features for a set of genes (e.g., top 14 ranked features), are shown in TABLE 7.

TABLE 7
Coefficients for Gene Variant Features related
to a Esophageal Source Tissue of Origin

| Feature | Coefficient Value |
|---|---|
| TP53 | 38 |
| SPEN | 31 |
| NUP93 | 38 |
| LRP1B | 54 |
| FYN | 35.5 |
| FOXO1 | 36 |
| ERCC3 | 49.5 |
| ERBB4 | 73 |
| EGFR | 42 |
| DOT1L | 40 |
| BRCA1 | 29 |
| ASXL2 | 31 |
| ARID1A | 37 |
| APC | 32 |

As such, in relation to outputting a prediction according to embodiments of method steps described above, the processing system can generate a prediction of esophageal tissue as the tissue source of origin upon evaluating values of the set of features corresponding to one or more, two or more, three or more, four or more, five or more, eight or more, or ten or more, of a set of small variant features listed in TABLE 7. In some examples, a gene panel (e.g., targeted sequencing panel for generating a prediction of esophogeal tissue source of origin) can include genes and/or gene features corresponding to the one or more, two or more, three or more, four or more, five or more, eight or more, or ten or more gene features listed in TABLE 7.

5.6. Example Gastric Tissue Source of Origin Coefficients

An example of model coefficient outputs for features associated with different genes, in relation to a prediction of a gastric tissue source of origin, and representative coefficient values, corresponding to small variant features for a set of genes (e.g., top 14 ranked features), are shown in TABLE 8.

TABLE 8
Coefficients for Gene Variant Features related
to a Gastric Tissue Source of Origin

| Feature | Coefficient Value |
|---|---|
| TP53 | 14 |
| SMAD4 | 18 |
| RHOA | 13 |
| PHOX2B | 11 |
| NOTCH1 | −3 |
| LMAP1 | 4.5 |
| KRAS | 72 |
| INPP4B | 13 |
| FLCN | 12.5 |
| FANCA | 13 |
| ERBB2 | 9.5 |
| DNMT1 | 51.5 |
| CTNNB1 | 9 |
| CDK12 | 3 |

As such, in relation to outputting a prediction according to embodiments of method steps described above, the processing system can generate a prediction of gastric tissue as the tissue source of origin upon evaluating values of the set of features corresponding to one or more, two or more, three or more, four or more, five or more, eight or more, or ten or more, of a set of small variant features listed in TABLE 8. In some examples, a gene panel (e.g., targeted sequencing panel for generating a prediction of gastric tissue source of origin) can include genes and/or gene features corresponding to the one or more, two or more, three or more, four or more, five or more, eight or more, or ten or more gene features listed in TABLE 8.

5.7. Example Head/Neck Tissue Source of Origin Coefficients

An example of model coefficient outputs for features associated with different genes, in relation to a prediction of a head/neck tissue source of origin, and representative coefficient values, corresponding to small variant features for a set of genes (e.g., top 14 ranked features), are shown in TABLE 9.

TABLE 9
Coefficients for Gene Variant Features related
to a Head/Neck Tissue Source of Origin

| Feature | Coefficient Value |
|---|---|
| ZRSR2 | 46 |
| SPTA1 | 39 |
| RUNX1T1 | 33 |
| PTPRT | 33.5 |
| PIK3CB | 51 |
| PBRM1 | 44 |
| NPM1 | 31.5 |
| NOTCH1 | 64 |
| MGA | 68 |
| KMT2D | 47 |
| KLH6 | 52 |
| GPR124 | 53 |
| FGFR3 | 36 |
| CASP8 | 43 |

As such, in relation to outputting a prediction according to embodiments of method steps described above, the processing system can generate a prediction of head/neck tissue as the tissue source of origin upon evaluating values of the set of features corresponding to one or more, two or more, three or more, four or more, five or more, eight or more, or ten or more, of a set of small variant features listed in TABLE 9. In some examples, a gene panel (e.g., targeted sequencing panel for generating a prediction of head/neck tissue source of origin) can include genes and/or gene features corresponding to the one or more, two or more, three or more, four or more, five or more, eight or more, or ten or more gene features listed in TABLE 9.

5.8. Example Hepatobiliary Tissue Source of Origin Coefficients

An example of model coefficient outputs for features associated with different genes, in relation to a prediction of a hepatobiliary tissue source of origin, and representative coefficient values, corresponding to small variant features for a set of genes (e.g., top 14 ranked features), are shown in TABLE 10.

TABLE 10
Coefficients for Gene Variant Features related
to a Hepatobiliary Tissue Source of Origin

| Feature | Coefficient Value |
|---|---|
| TSHR | 53 |
| TP53 | 46 |
| SMARCD1 | 33 |
| SLIT2 | 56 |
| RPTOR | 38 |
| NTRK2 | 18 |
| MSH6 | 29 |
| MCL1 | 17 |
| DNAJB1 | 16 |
| CTNNB1 | 85 |
| CTCF | 37 |
| CJD2 | 34 |
| CCNE1 | 88 |
| ARID1A | 27 |

As such, in relation to outputting a prediction according to embodiments of method steps described above, the processing system can generate a prediction of hepatobiliary tissue as the tissue source of origin upon evaluating values of the set of features corresponding to one or more, two or more, three or more, four or more, five or more, eight or more, or ten or more, of a set of small variant features listed in TABLE 10. In some examples, a gene panel (e.g., targeted sequencing panel for generating a prediction of hepatobiliary tissue source of origin) can include genes and/or gene features corresponding to the one or more, two or more, three or more, four or more, five or more, eight or more, or ten or more gene features listed in TABLE 10.

5.9. Example Leukemia Source of Origin Coefficients

An example of model coefficient outputs for features associated with different genes, in relation to a prediction of a leukemia source of origin, and representative coefficient values, corresponding to small variant features for a set of genes (e.g., top 13 ranked features), are shown in TABLE 11.

TABLE 11
Coefficients for Gene Variant Features
related to a Leukemia Source of Origin

| Feature | Coefficient Value |
|---|---|
| TP53 | −15.5 |
| RUNX1 | 0 |
| PIK3CA | 0 |
| PGR | 12.5 |
| LRP1B | −.5 |
| KRAS | −4 |
| IRS1 | 22.5 |
| IDH1 | 24.5 |
| ERBB2 | 7.5 |
| DNMT3A | 34 |
| CSF1R | 5.5 |
| ASXL1 | 5.5 |
| ACVR1B | 7.5 |

As such, in relation to outputting a prediction according to embodiments of method steps described above, the processing system can generate a prediction of leukemia as the source of origin upon evaluating values of the set of features corresponding to one or more, two or more, three or more, four or more, five or more, eight or more, or ten or more, of a set of small variant features listed in TABLE 11. In some examples, a gene panel (e.g., targeted sequencing panel for generating a prediction of leukemia source of origin) can include genes and/or gene features corresponding to the one or more, two or more, three or more, four or more, five or more, eight or more, or ten or more gene features listed in TABLE 11.

5.10. Example Lung Tissue Source of Origin Coefficients

An example of model coefficient outputs for features associated with different genes, in relation to a prediction of a lung tissue source of origin, and representative coefficient values, corresponding to small variant features for a set of genes (e.g., top 14 ranked features), are shown in TABLE 12. For example, as shown in TABLE 12 below, presence of LRP1B variants provides positive evidence for a lung cancer type, which is consistent for instance with TABLE 4 above, in which the coefficient for LRP1B variants was strongly negative in relation to a breast cancer type.

TABLE 12
Coefficients for Gene Variant Features related
to a Lung Tissue Source of Origin

| Feature | Coefficient Value |
|---|---|
| TET2 | 45 |
| SPTA1 | 82.5 |
| SMARCA4 | 45 |
| POLE | 48 |
| LRP1B | 113 |
| KEAP1 | 89 |
| IRF4 | 55 |
| IL7R | 44.5 |
| IKZF1 | 62 |
| H3F3A | 50 |
| GRM3 | 56 |
| CDKN2A | 54 |
| BCORL1 | 50 |
| ARID2 | 53 |

As such, in relation to outputting a prediction according to embodiments of method steps described above, the processing system can generate a prediction of lung tissue as the tissue source of origin upon evaluating values of the set of features corresponding to one or more, two or more, three or more, four or more, five or more, eight or more, or ten or more, of a set of small variant features listed in TABLE 12. In some examples, a gene panel (e.g., targeted sequencing panel for generating a prediction of lung tissue source of origin) can include genes and/or gene features corresponding to the one or more, two or more, three or more, four or more, five or more, eight or more, or ten or more gene features listed in TABLE 12.

5.11. Example Lymphoma Source of Origin Coefficients

An example of model coefficient outputs for features associated with different genes, in relation to a prediction of a lymphoma source of origin, and representative coefficient values, corresponding to small variant features for a set of genes (e.g., top 14 ranked features), are shown in TABLE 13.

TABLE 13
Coefficients for Gene Variant Features
related to a Lymphoma Source of Origin

| Feature | Coefficient Value |
|---|---|
| TP53 | −28 |
| TNFRSF14 | 60 |
| SOCS1 | 100 |
| REL | 32 |
| NTRK2 | 29 |
| MYD88 | 57 |
| KMT2D | 48 |
| KAT6A | 37 |
| HIST1H1C | 28 |
| FOXO1 | 26 |
| CREBBP | 90 |
| BCR | 49 |
| BCL2 | 35 |
| AMER1 | 26 |

As such, in relation to outputting a prediction according to embodiments of method steps described above, the processing system can generate a prediction of lymphoma as the tissue source of origin upon evaluating values of the set of features corresponding to one or more, two or more, three or more, four or more, five or more, eight or more, or ten or more, of a set of small variant features listed in TABLE 13. In some examples, a gene panel (e.g., targeted sequencing panel for generating a prediction of lymphoma source of origin) can include genes and/or gene features corresponding to the one or more, two or more, three or more, four or more, five or more, eight or more, or ten or more gene features listed in TABLE 13.

5.12. Example Melanoma Source of Origin Coefficients

An example of model coefficient outputs for features associated with different genes, in relation to a prediction of a melanoma source of origin, and representative coefficient values, corresponding to small variant features for a set of genes (e.g., top 11 ranked features), are shown in TABLE 14.

TABLE 14
Coefficients for Gene Variant Features
related to a Melanoma Source of Origin

| Feature | Coefficient Value |
|---|---|
| VTCN1 | 12.5 |
| TP53 | 2.7 |
| SNCAIP | 2.4 |
| PIK3CA | 0 |
| NTRK1 | 10.2 |
| LRP1B | −.3 |
| KRAS | −3 |
| ERBB2 | 13 |
| EPHA5 | 4 |
| EPHA3 | 17.5 |
| DNMT3B | 23 |

As such, in relation to outputting a prediction according to embodiments of method steps described above, the processing system can generate a prediction of melanoma tissue as the tissue source of origin upon evaluating values of the set of features corresponding to one or more, two or more, three or more, four or more, five or more, eight or more, or ten or more, of a set of small variant features listed in TABLE 14. In some examples, a gene panel (e.g., targeted sequencing panel for generating a prediction of melanoma source of origin) can include genes and/or gene features corresponding to the one or more, two or more, three or more, four or more, five or more, eight or more, or ten or more gene features listed in TABLE 14.

5.13. Example Multiple Myeloma Source of Origin Coefficients

An example of model coefficient outputs for features associated with different genes, in relation to a prediction of a multiple myeloma source of origin, representative coefficient values, corresponding to small variant features for a set of genes (e.g., top 14 ranked features), are shown in TABLE 15.

TABLE 15
Coefficients for Gene Variant Features related
to a M. Myeloma Source of Origin

| Feature | Coefficient Value |
|---|---|
| SPTA1 | −9 |
| SLIT2 | 26 |
| SHQ1 | 13 |
| RAF1 | 11 |
| NRAS | 30 |
| IDH2 | 58 |
| FUBP1 | 61 |
| FAM46C | 25 |
| ERBB4 | 29 |
| EIF1AX | 65 |
| CD74 | 28 |
| BTG1 | 29 |
| BRAF | 103 |
| APC | 23 |

As such, in relation to outputting a prediction according to embodiments of method steps described above, the processing system can generate a prediction of multiple myeloma as the source of origin upon evaluating values of the set of features corresponding to one or more, two or more, three or more, four or more, five or more, eight or more, or ten or more, of a set of small variant features listed in TABLE 15. In some examples, a gene panel (e.g., targeted sequencing panel for generating a prediction of multiple myeloma source of origin) can include genes and/or gene features corresponding to the one or more, two or more, three or more, four or more, five or more, eight or more, or ten or more gene features listed in TABLE 15.

5.14. Example Non-Cancer Grouping Coefficients

An example of model coefficient outputs for features associated with different genes, in relation to a prediction of a non-cancer grouping, and representative coefficient values, corresponding to small variant features for a set of genes (e.g., top 14 ranked features), are shown in TABLE 16. For example, as shown in TABLE 16, presence of TP53 variants provide positive evidence for cancer, as demonstrated with its strong negative coefficient in relation to non-cancer, while presence of KRAS variants provide positive evidence that the sample is probably not harmless and should be grouped with the cancer grouping.

TABLE 16
Coefficients for Gene Variant Features
related to a Non-Cancer Grouping

| Feature | Coefficient Value |
|---|---|
| TP53 | −141 |
| TET2 | −30 |
| PTPRT | −37.5 |
| PIK3CA | −67 |
| NOTCH1 | −37 |
| MGA | −33 |
| LRP1B | −65 |
| KRAS | −92 |
| ERBB4 | −33 |
| ERBB2 | −32 |
| CTNNB1 | −33 |
| BRAF | −34 |
| ATR | −34 |
| APC | −32 |

As such, in relation to outputting a prediction according to embodiments of method steps described above, the processing system can generate a prediction of cancer/non-cancer upon evaluating values of the set of features corresponding to one or more, two or more, three or more, four or more, five or more, eight or more, or ten or more, of a set of small variant features listed in TABLE 16. In some examples, a gene panel (e.g., targeted sequencing panel for generating a prediction of cancer/non-cancer) can include genes and/or gene features corresponding to the one or more, two or more, three or more, four or more, five or more, eight or more, or ten or more gene features listed in TABLE 16.

5.15. Example Ovarian Tissue Source of Origin Coefficients

An example of model coefficient outputs for features associated with different genes, in relation to a prediction of an ovarian tissue source of origin, and representative coefficient values, corresponding to small variant features for a set of genes (e.g., top 14 ranked features), are shown in TABLE 17.

TABLE 17
Coefficients for Gene Variant Features related
to an Ovarian Tissue Source of Origin

| Feature | Coefficient Value |
|---|---|
| TP53 | 53 |
| TNFRSF14 | 37.5 |
| RUNX1 | 14 |
| PIK3CD | 38 |
| PAX8 | 14 |
| NUTM1 | 31 |
| MSH2 | 25 |
| MAP3K1 | 38 |
| KLF4 | 31 |
| FAT1 | 13 |
| FANCC | 34 |
| ERCC4 | 38 |
| ATR | 95 |
| ARID1B | −14 |

As such, in relation to outputting a prediction according to embodiments of method steps described above, the processing system can generate a prediction of ovarian tissue as the tissue source of origin upon evaluating values of the set of features corresponding to one or more, two or more, three or more, four or more, five or more, eight or more, or ten or more, of a set of small variant features listed in TABLE 17. In some examples, a gene panel (e.g., targeted sequencing panel for generating a prediction of ovarian tissue source of origin) can include genes and/or gene features corresponding to the one or more, two or more, three or more, four or more, five or more, eight or more, or ten or more gene features listed in TABLE 17.

5.16. Example Pancreatic Tissue Source of Origin Coefficients

An example of model coefficient outputs for features associated with different genes, in relation to a prediction of a pancreatic tissue source of origin, and representative coefficient values, corresponding to small variant features for a set of genes (e.g., top 14 ranked features), are shown in TABLE 18.

TABLE 18
Coefficients for Gene Variant Features related
to a Pancreatic Tissue Source of Origin

| Feature | Coefficient Value |
|---|---|
| U2AF1 | 32 |
| TP53 | 30 |
| TGFBR | 23 |
| SMAD4 | 16 |
| NOTCH1 | 23 |
| LZTR1 | 25 |
| KRAS | 118 |
| KMT2D | 32 |
| FANCE | 32 |
| FANCA | −24.5 |
| DNMT1 | −48 |
| CDKN2A | 16 |
| ARID1B | 25 |
| APC | −17 |

As such, in relation to outputting a prediction according to embodiments of method steps described above, the processing system can generate a prediction of pancreatic tissue as the tissue source of origin upon evaluating values of the set of features corresponding to one or more, two or more, three or more, four or more, five or more, eight or more, or ten or more, of a set of small variant features listed in TABLE 18. In some examples, a gene panel (e.g., targeted sequencing panel for generating a prediction of pancreatic tissue source of origin) can include genes and/or gene features corresponding to the one or more, two or more, three or more, four or more, five or more, eight or more, or ten or more gene features listed in TABLE 18.

5.17. Example Prostate Tissue Source of Origin Coefficients

An example of model coefficient outputs for features associated with different genes, in relation to a prediction of a prostate tissue source of origin, and representative coefficient values, corresponding to small variant features for a set of genes (e.g., top 14 ranked features), are shown in TABLE 19.

TABLE 19
Coefficients for Gene Variant Features related
to a Prostate Tissue Source of Origin

| Feature | Coefficient Value |
|---|---|
| TP53 | −50 |
| PTPRT | −10 |
| PIK3CA | −16 |
| NOTCH1 | −8 |
| MGA | 24.5 |
| LRP1B | −14 |
| KRAS | −36 |
| KMT2D | −10 |
| INPP4B | 12 |
| GRIN2A | 33 |
| ERBB4 | −13 |
| BRAF | −8.5 |
| ATR | −8 |
| APC | −10 |

As such, in relation to outputting a prediction according to embodiments of method steps described above, the processing system can generate a prediction of prostate tissue as the tissue source of origin upon evaluating values of the set of features corresponding to one or more, two or more, three or more, four or more, five or more, eight or more, or ten or more, of a set of small variant features listed in TABLE 19. In some examples, a gene panel (e.g., targeted sequencing panel for generating a prediction of prostate tissue source of origin) can include genes and/or gene features corresponding to the one or more, two or more, three or more, four or more, five or more, eight or more, or ten or more gene features listed in TABLE 19.

5.18. Example Renal Tissue Source of Origin Coefficients

An example of model coefficient outputs for features associated with different genes, in relation to a prediction of a renal tissue source of origin, and representative coefficient values, corresponding to small variant features for a set of genes (e.g., top 14 ranked features), are shown in TABLE 20.

TABLE 20
Coefficients for Gene Variant Features related
to a Renal Tissue Source of Origin

| Feature | Coefficient Value |
|---|---|
| TSC1 | 48 |
| TET1 | 32.5 |
| SUZ12 | 22.5 |
| SNCAIP | 32.5 |
| SMARCD1 | 16.5 |
| SDHA | 22 |
| PBRM1 | 24 |
| NTRK1 | 27 |
| NOTCH1 | 54 |
| MST1R | 39 |
| ERCC2 | 26 |
| ERBB2 | 17.5 |
| EP300 | 30 |
| BCL6 | 22 |

As such, in relation to outputting a prediction according to embodiments of method steps described above, the processing system can generate a prediction of renal tissue as the tissue source of origin upon evaluating values of the set of features corresponding to one or more, two or more, three or more, four or more, five or more, eight or more, or ten or more, of a set of small variant features listed in TABLE 20. In some examples, a gene panel (e.g., targeted sequencing panel for generating a prediction of renal tissue source of origin) can include genes and/or gene features corresponding to the one or more, two or more, three or more, four or more, five or more, eight or more, or ten or more gene features listed in TABLE 20.

5.19. Example Thyroid Tissue Source of Origin Coefficients

An example of model coefficient outputs for features associated with different genes, in relation to a prediction of a thyroid tissue source of origin, and representative coefficient values, corresponding to small variant features for a set of genes (e.g., top 10 ranked features), are shown in TABLE 21.

TABLE 21
Coefficients for Gene Variant Features related
to a Thyroid Tissue Source of Origin

| Feature | Coefficient Value |
|---|---|
| ZFHX3 | 1 |
| TP53 | −7.5 |
| RHOA | 11 |
| PIK3CA | −1 |
| LRP1B | −1.5 |
| KRAS | −4.5 |
| ERBB4 | −0.5 |
| EGFR | 0.5 |
| BRAF | 16 |
| APC | −0.3 |

As such, in relation to outputting a prediction according to embodiments of method steps described above, the processing system can generate a prediction of thyroid tissue as the tissue source of origin upon evaluating values of the set of features corresponding to one or more, two or more, three or more, four or more, five or more, eight or more, or ten or more, of a set of small variant features listed in TABLE 21. In some examples, a gene panel (e.g., targeted sequencing panel for generating a prediction of thyroid tissue source of origin) can include genes and/or gene features corresponding to the one or more, two or more, three or more, four or more, five or more, eight or more, or ten or more gene features listed in TABLE 21.

5.20. Example Uterine Tissue Source of Origin Coefficients

An example of model coefficient outputs for features associated with different genes, in relation to a prediction of an uterine tissue source of origin, and representative coefficient values, corresponding to small variant features for a set of genes (e.g., top 14 ranked features), are shown in TABLE 22.

TABLE 22
Coefficients for Gene Variant Features related
to a Uterine Tissue Source of Origin

| Feature | Coefficient Value |
|---|---|
| TP53 | −25 |
| TET1 | 25 |
| SMARCA4 | 9.5 |
| RB1 | 24 |
| RAD21 | 10 |
| PTPRT | 15 |
| PTPN11 | 12 |
| KRAS | −11 |
| IRS2 | 14 |
| EPHB1 | 21 |
| EPHA5 | 14.5 |
| EED | 14 |
| CDC73 | 42 |
| ASXL1 | 20 |

As such, in relation to outputting a prediction according to embodiments of method steps described above, the processing system can generate a prediction of uterine tissue as the tissue source of origin upon evaluating values of the set of features corresponding to one or more, two or more, three or more, four or more, five or more, eight or more, or ten or more, of a set of small variant features listed in TABLE 22. In some examples, a gene panel (e.g., targeted sequencing panel for generating a prediction of uterine tissue source of origin) can include genes and/or gene features corresponding to the one or more, two or more, three or more, four or more, five or more, eight or more, or ten or more gene features listed in TABLE 22.

5.21. Example Precision and Recall Metrics for Tissue Sources of Origin Predictions

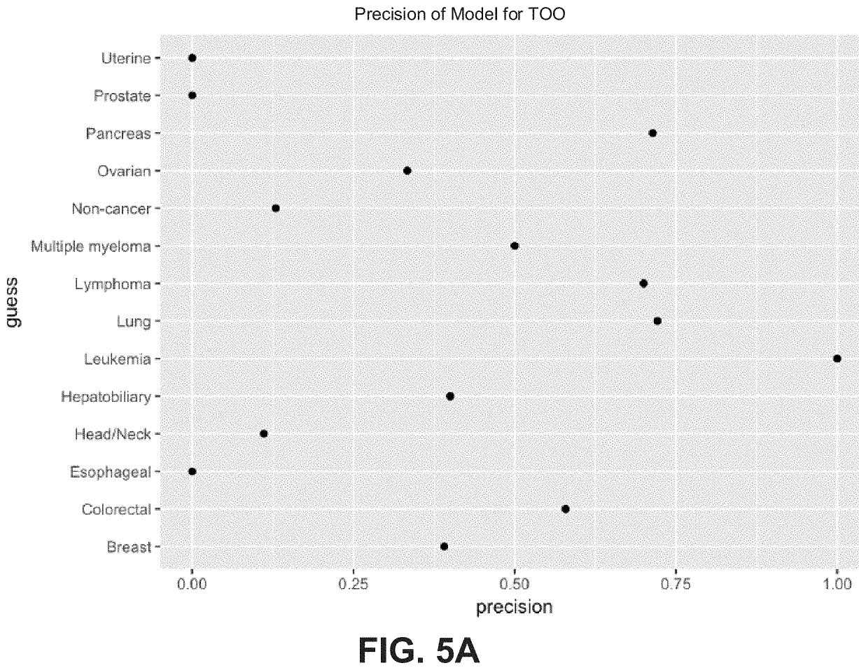

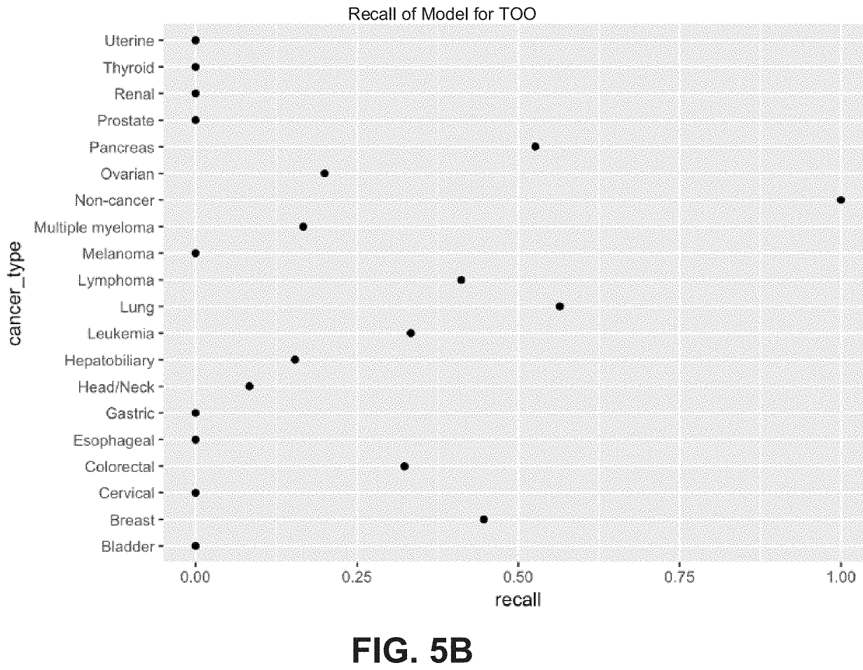

FIG. 5A depicts an example of precision metric outputs of a predictive model, in relation to predictions of a portion of the tissue sources of origin shown in TABLES 1-22, where metric outputs were determined from a sample data set and a training data set from at least 8 cfDNA samples per tissue source of origin. In more detail, FIG. 5A includes a plot of precision, a fraction of samples classified with a given tissue source of origin that are actually of that tissue source of origin, thereby characterizing a fraction of true positives to total positives determined for each tissue source. For instance, FIG. 5A shows that approximately 70% of the samples classified by the prediction model as lymphoma are actually lymphoma samples, while approximately 50% of the samples classified by the prediction model as multiple myeloma are actually multiple myeloma samples.

In generating and/or returning a prediction after processing a set of features with an embodiment of the prediction model described above, the processing subsystem can output a tissue source corresponding to the set of features and satisfying a precision condition during training of the prediction model, the precision condition evaluated across cfDNA samples of a population of subjects. The precision condition can have a first condition value in a training subject population associated with development of the prediction model, and a second condition value in an in-use subject population associated with use of the prediction model, thereby providing different precision conditions in training of the prediction model as compared to use of the prediction model.

FIG. 5B depicts an example of recall metric outputs of a predictive model, in relation to predictions of a portion of the tissue sources of origin shown in TABLES 1-22. In more detail, FIG. 5B includes a plot of recall, a fraction of samples that are of a tissue source of origin that are actually classified with that tissue source of origin, thereby characterizing a fraction of true positives to a total of true positives and false negatives determined for each tissue source. For instance, FIG. 5B shows that approximately ⅓ of actual leukemia samples were correctly classified by the prediction model as leukemia. In conjunction with FIG. 5A, it can be deduced that when the predictive model classified a sample as leukemia, that classification was correct (e.g., see FIG. 5A showing “Leukemia” at 100%), however approximately ⅔ of the remaining actual leukemia samples were classified under other cancer types.

In generating and/or returning a prediction after processing a set of features with an embodiment of the prediction model described above, the processing subsystem can output a candidate tissue source corresponding to the set of features and satisfying a recall condition during training of the prediction model, the recall condition evaluated across cfDNA samples of a population of subjects. The recall condition can have a first condition value in a training subject population associated with development of the prediction model, and a second condition value in an in-use subject population associated with use of the prediction model, thereby providing different recall conditions in training of the prediction model as compared to use of the prediction model. Furthermore, in relation to outputting a prediction according to embodiments of method steps described above, the processing system can generate a prediction of a tissue source of origin upon evaluating values of the set of features listed in one or more of any of the TABLES 2-22. For example, a gene panel (e.g., targeted sequencing panel) can include one or more genes and/or gene features listed in any of TABLES 2-22, and from any combination of such tables. Merely by way of example, a gene panel can include one or more, two more, three or more, four or more, five or more, eight or more, or ten or more, genes listed from each table of the one or more, two or more, three or more, four or more, five or more, eight or more, or ten or more, of TABLES 2-22.

### 6. Additional Example Prediction Model Coefficients for Different Tissue Sources of Origin

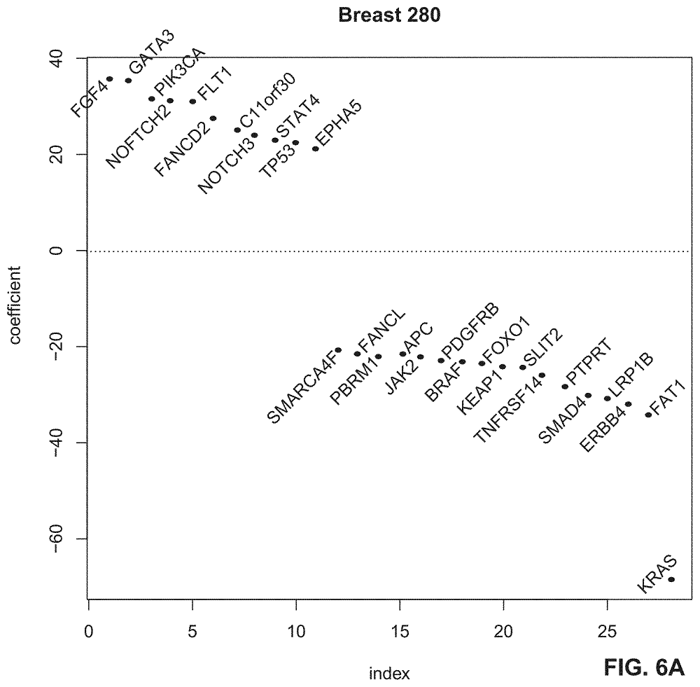

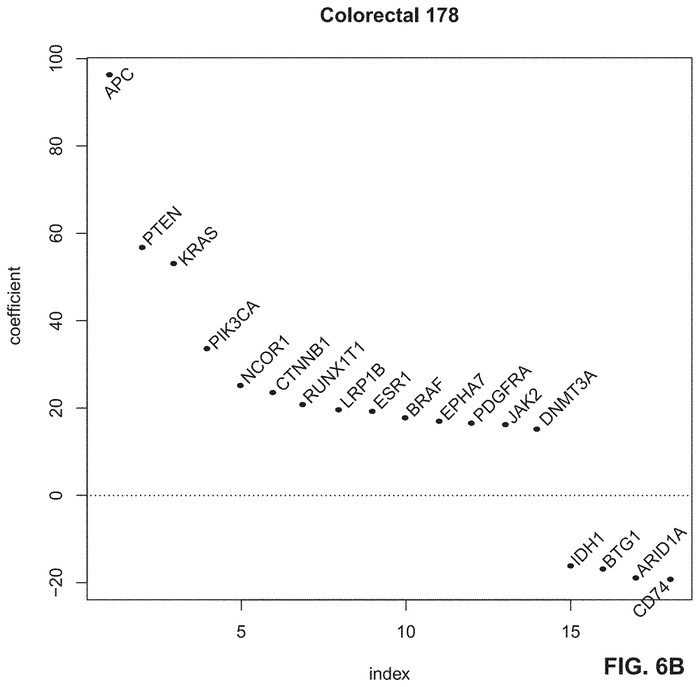

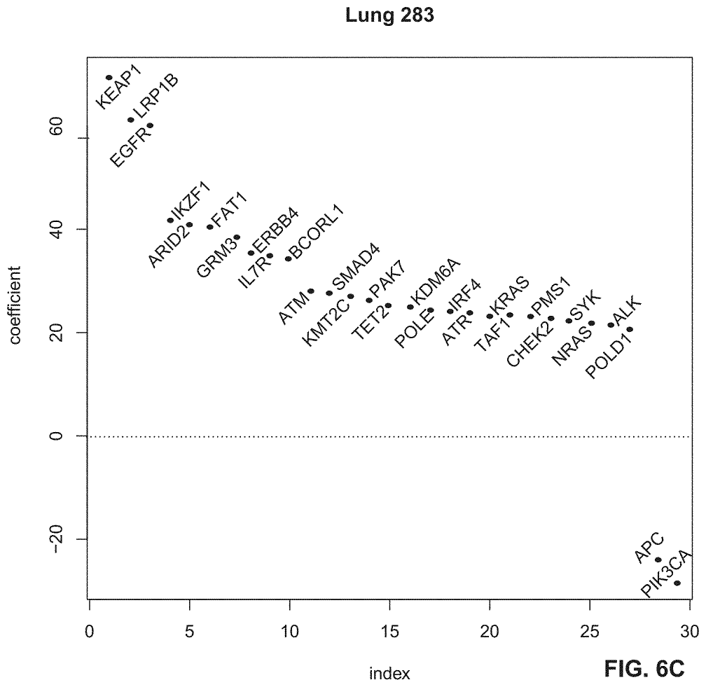

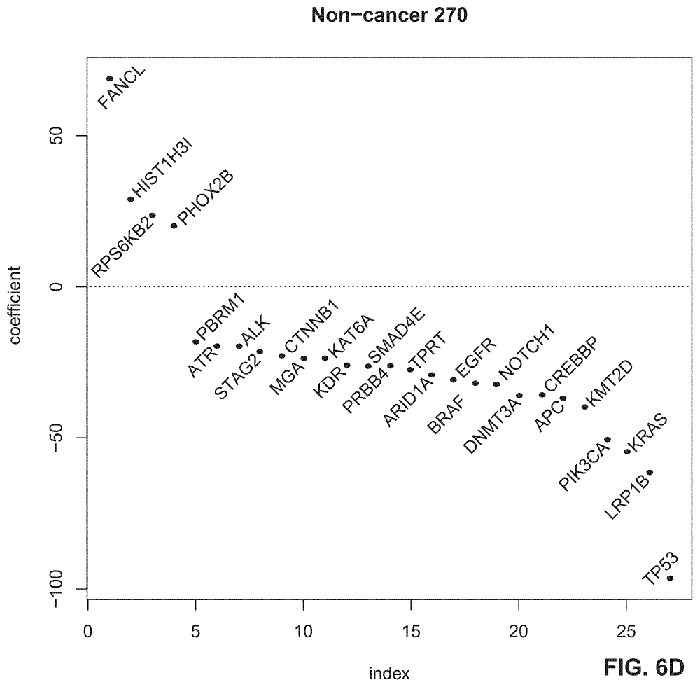

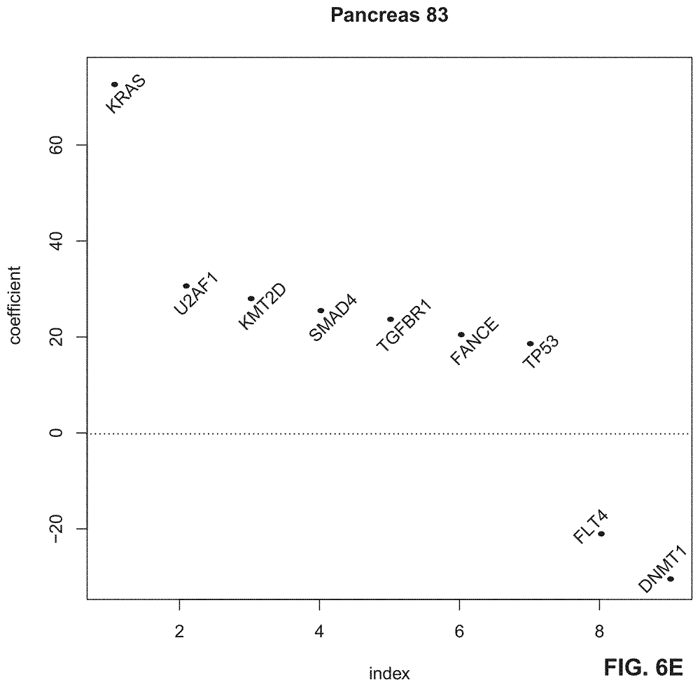

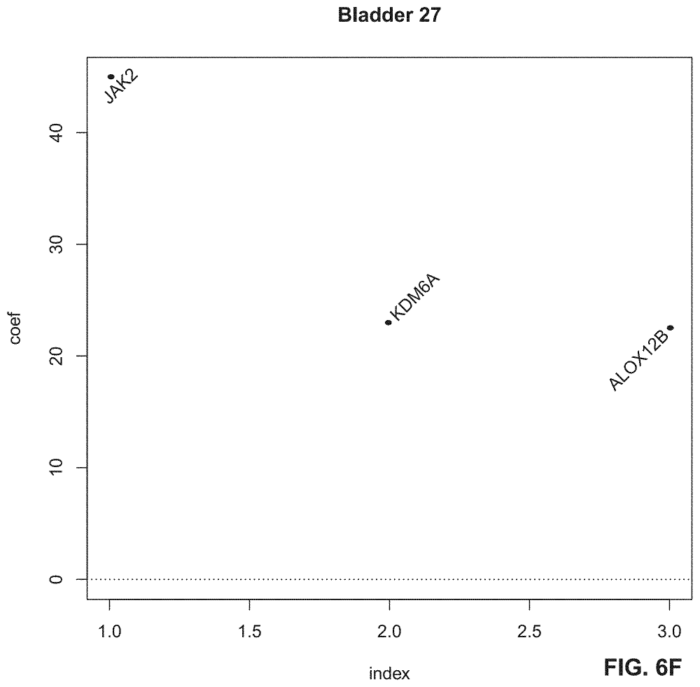

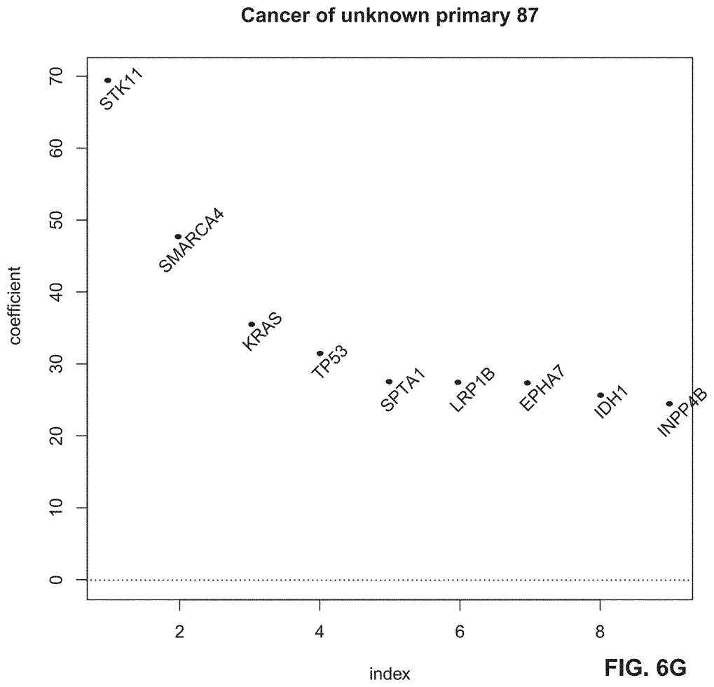

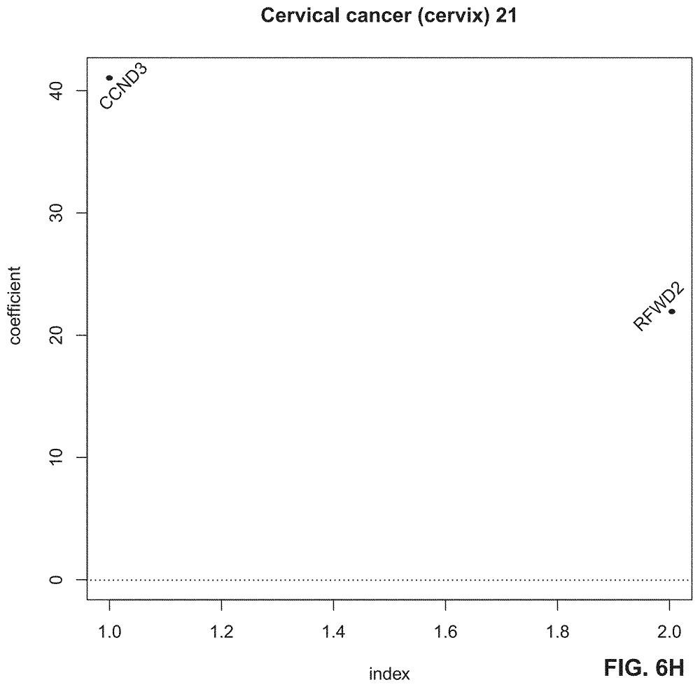

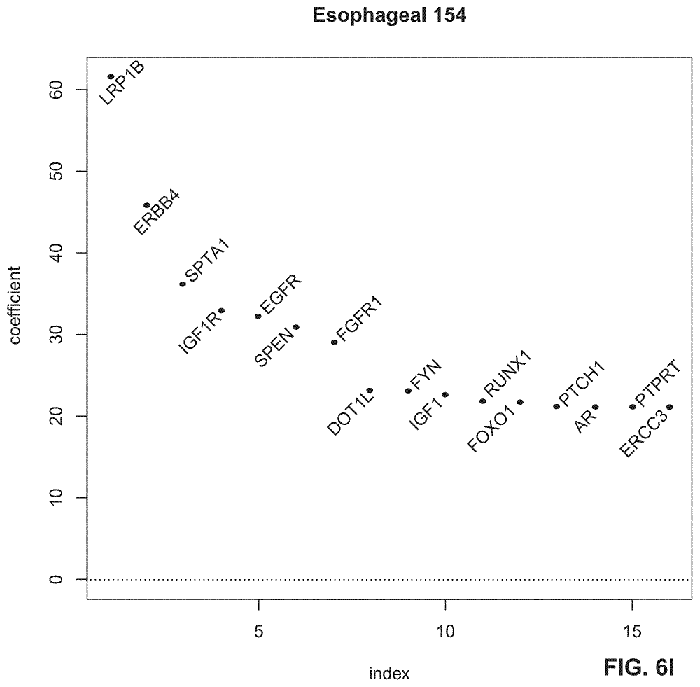

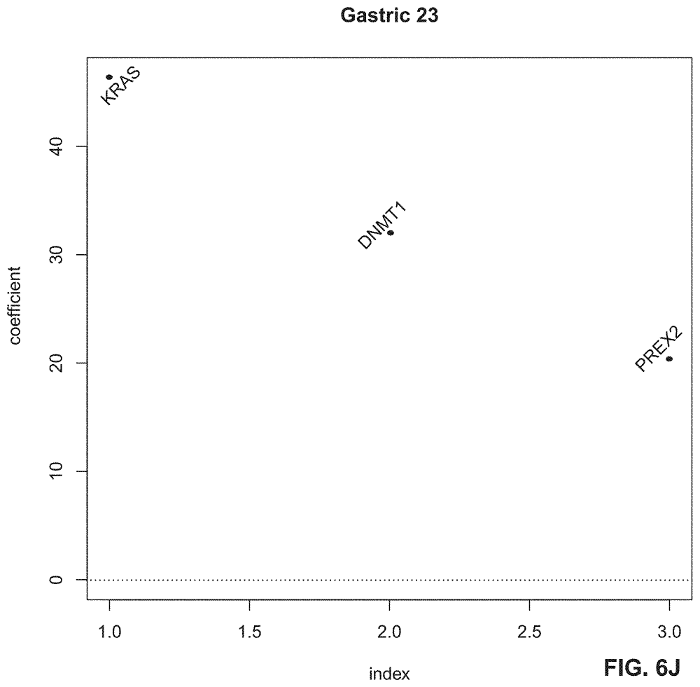

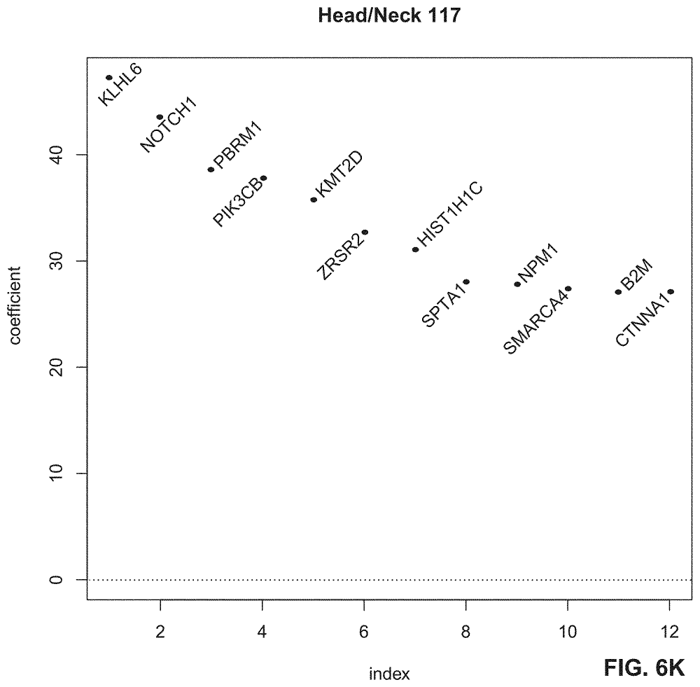

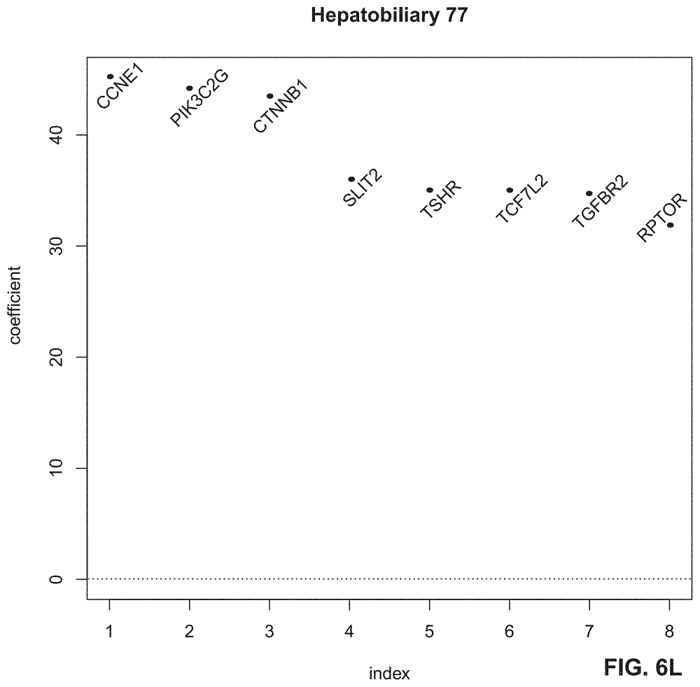

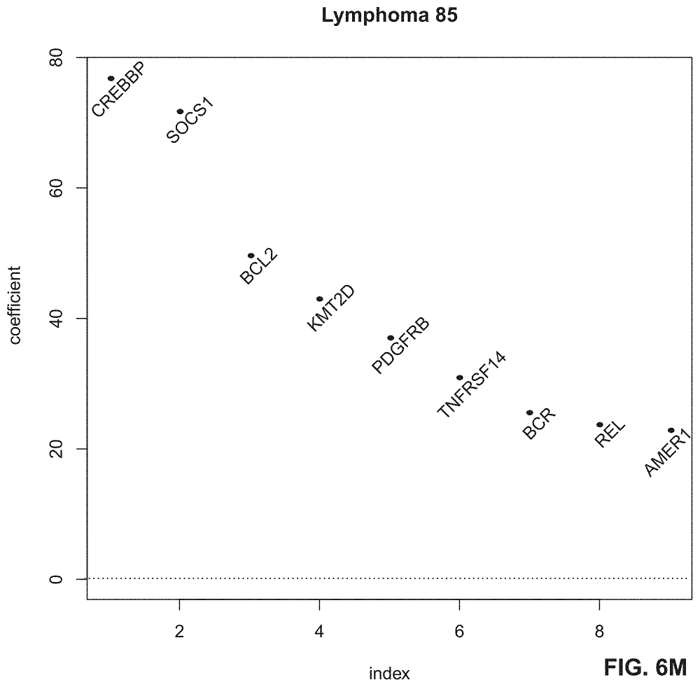

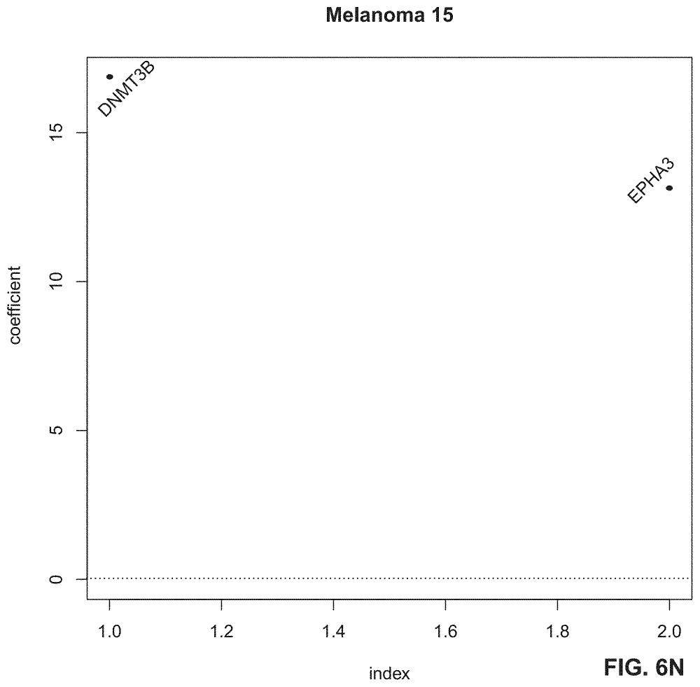

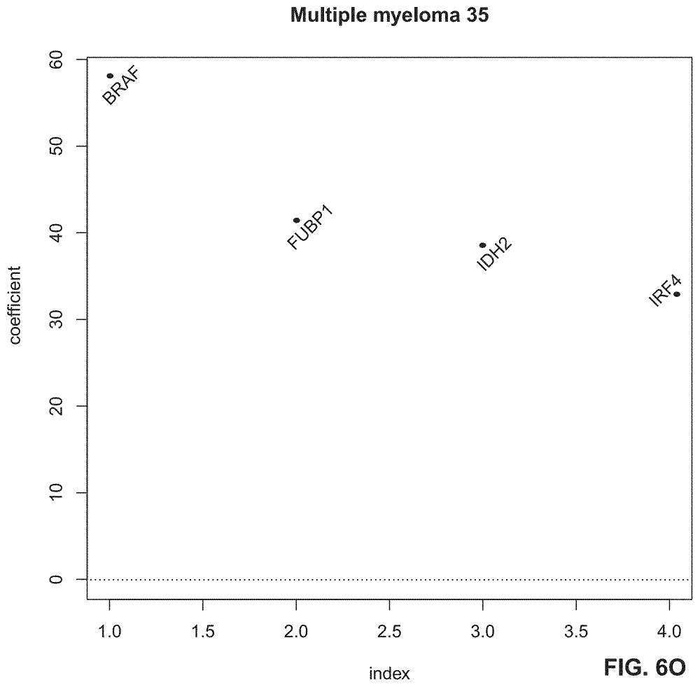

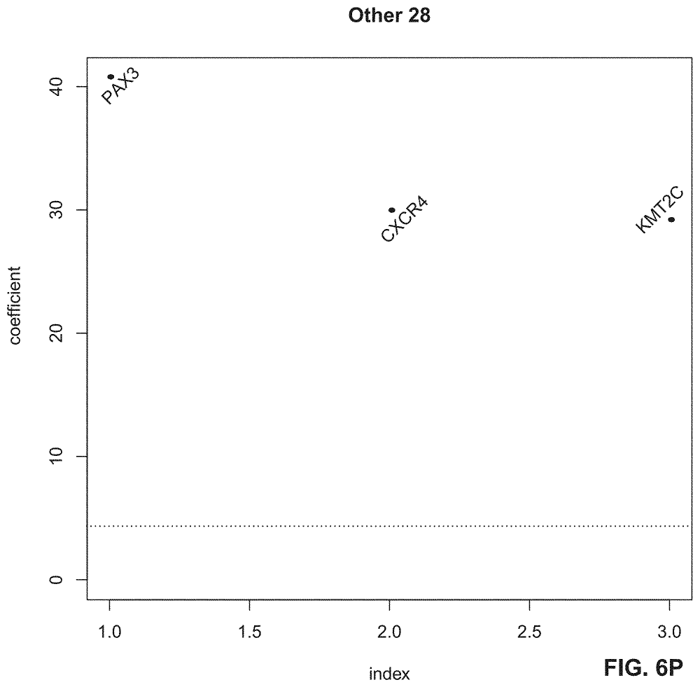

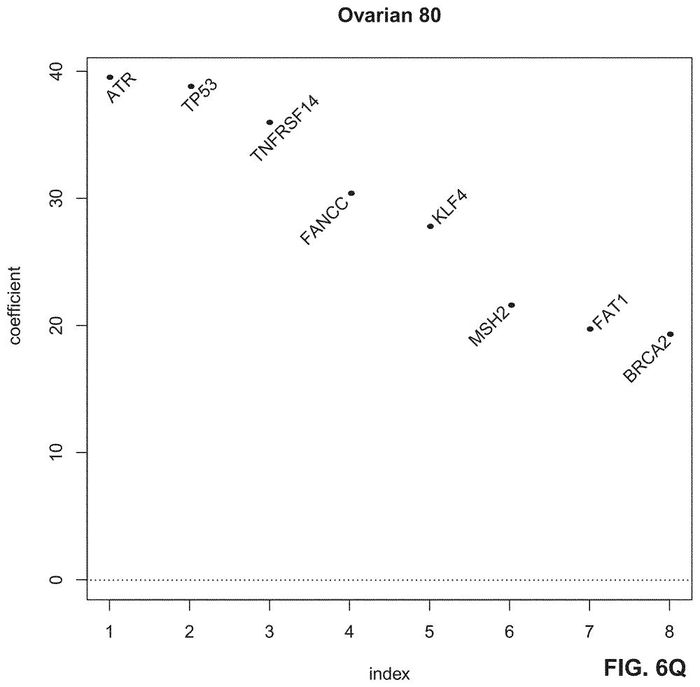

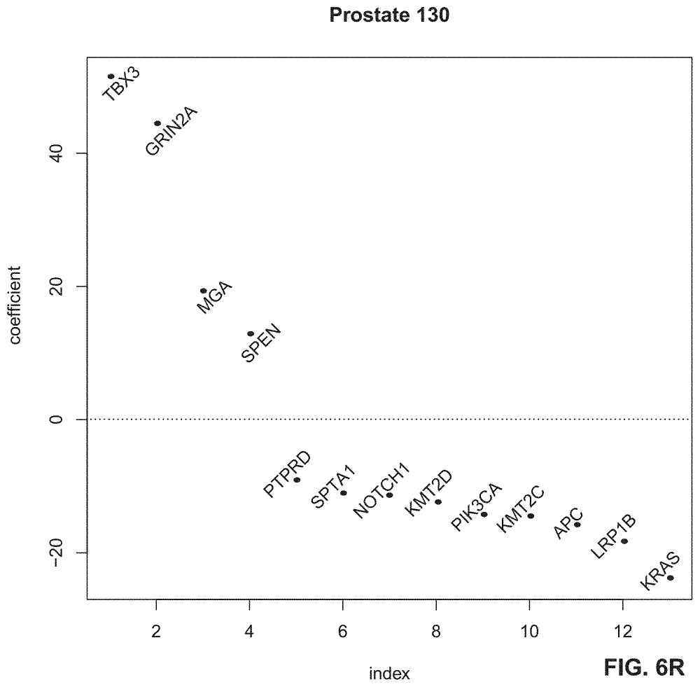

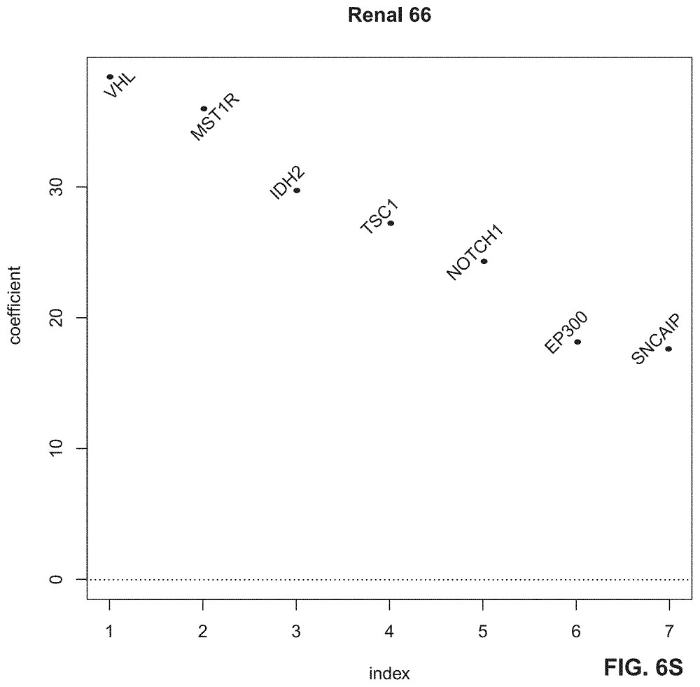

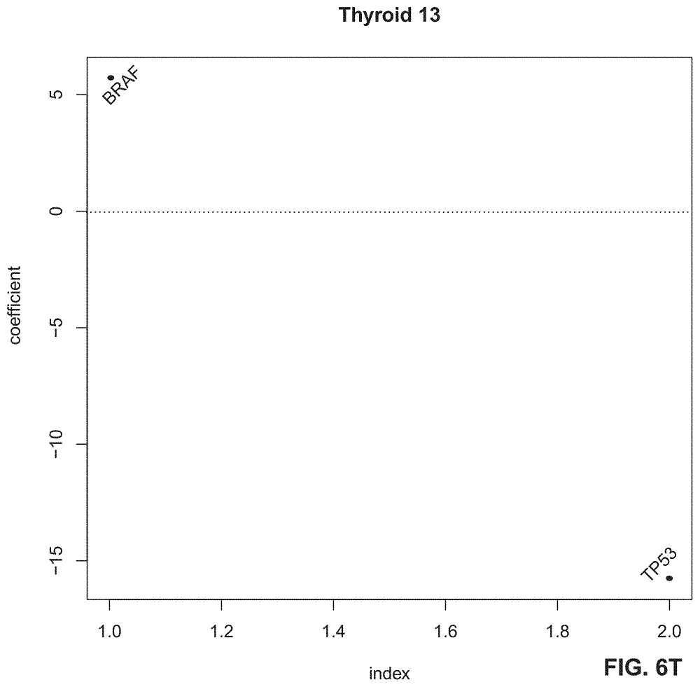

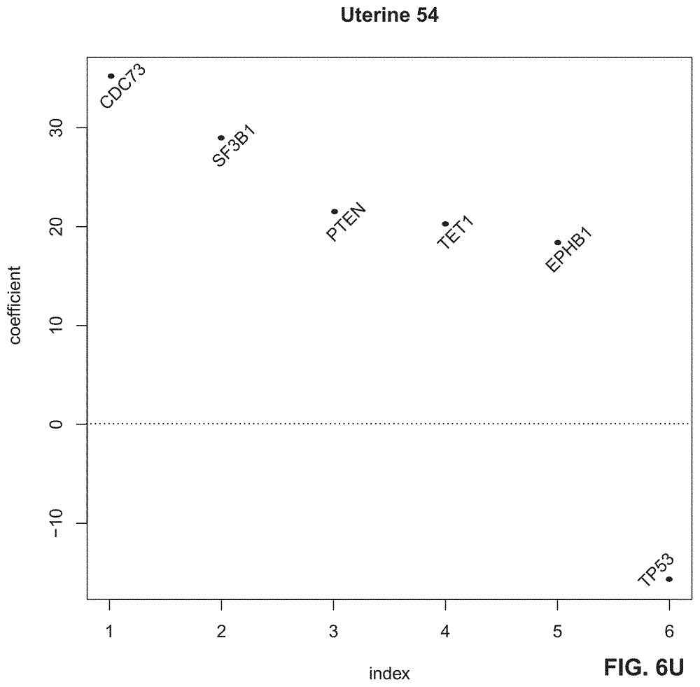

FIGS. 6A-6U depict another example of model coefficient outputs for features (e.g., small variant features) associated with different genes in relation to the prediction of multiple tissue sources of origin. The example model coefficients below were determined through training of a multinomial regression model using a training data set obtained from training samples. As shown in TABLE 23, the training samples (N=1435) were blood samples collected from individuals diagnosed with cancer (N=859) and healthy individuals with no cancer diagnosis (N=576). Cell-free DNA were extracted from the samples, sequenced, and analyzed for features (e.g., non-synonymous informative variants within a gene) to produce training data for the training data set. A breakdown of the cancer samples (N=859) by cancer type is provided in TABLE 24.

TABLE 23
Samples used for training.

| | N |
|---|---|
| Cancer | 859 |
| Non-cancer | 576 |
| Total samples | 1435 |

TABLE 24
Cancer samples by cancer type.

| | N |
|---|---|
| Bladder | 10 |
| Breast | 349 |
| Cancer of unknown primary | 10 |
| Cervical | 13 |
| Colorectal | 47 |
| Esophageal | 24 |
| Gastric | 11 |
| Head/Neck | 20 |
| Hepatobiliary | 15 |
| Leukemia | 0 |
| Lung | 122 |
| Lymphoma | 24 |
| Melanoma | 10 |
| Multiple myeloma | 11 |
| Other | 9 |
| Ovarian | 19 |
| Pancreas | 27 |
| Prostate | 71 |
| Renal | 27 |
| Thyroid | 13 |
| Uterine | 27 |

It is noted that while there is some overlap in the training samples used in this example and the training samples included in the previous example at TABLES 1-22, there are also some differences in the training data sets that, in some cases as demonstrated below, produced different model coefficients and/or gene features associated with the prediction of the tissue source of origin. Further differences between the present analyses at FIGS. 6A-6U and the previous analyses of TABLES 1-22 include differences in generating features, such as different analysis of what constitutes a “non-synonymous” informative variant within a gene, and different sets of cross-validation folds. For instance, the coefficients and gene features generated in the analysis of TABLES 1-22 used one set of cross-validation folds, while the coefficients and gene features generated in the analysis of FIGS. 6A-6U below used a different set of cross-validation folds, whereby a comparison across the two different sets of folds showed n=132 samples being equal, n=1280 samples not equal, and n=64 as not applicable for samples that were present in only one of the two folds.

FIG. 6A depicts another example of model coefficient outputs for features associated with different genes, in relation to a prediction of a breast tissue source of origin. As shown in FIG. 6A, a multinomial regression model can have coefficients corresponding to small variant features for different genes, in a regression between the small variant features and breast tissue against other tissue groups. Representative coefficient values are depicted in FIG. 6A, where positive coefficient values indicate evidence for a breast tissue source, in relation to tissue source of origin, and negative coefficient values indicate evidence for another type of cancer, in relation to tissue source of origin. For example, as shown in FIG. 6A, presence of a PIK3CA variant (positive coefficient) suggests that the tissue source of origin is breast cancer, while presence of APC variant (negative coefficient) suggests that the tissue source of origin is not breast cancer. In general, detection of variants in genes including FGF4, GATA3, PIK3CA, NOTCH2, FLT1, FANCD2, C11orf30, NOTCH3, STAT4, TP53, and EPHA5 provide positive evidence for a breast tissue source of origin, while detection of variants in genes including SMARCA4, FANCL, PBRM1, APC, JAK2, PDGFRB, BRAF, FOXO1, KEAP1, SLIT2, TNFRSF14, PTPRT, SMAD4, LRP1B, ERBB1, and FAT1 provide negative evidence for a breast tissue source of origin.

FIG. 6B depicts an example of model coefficient outputs (e.g., representative coefficient values) for features associated with different genes, in relation to a prediction of a colorectal tissue source of origin. For example, as shown in FIG. 6B, presence of APC variants (positive coefficient) increase the estimated probability that the tissue of origin is colorectal. In general, detection of variants in genes including APC, PTEN, KRAS, PIK3CA, NCOR1, CTNNB1, RUNX1T1, LRP1B, ESR1, BRAF, EPHA7, PDGFRA, JAK2, and DNMT3A provide positive evidence for a colorectal tissue source of origin, while detection of variants in genes including IDH1, BTG1, ARID1A, and CD74 provide negative evidence for a colorectal tissue source of origin.

FIG. 6C depicts an example of model coefficient outputs for features associated with different genes, in relation to a prediction of a lung tissue source of origin. For example, as shown in FIG. 6C, presence of KEAP1, LRP1B, and/or EGFR variants can suggest that the tissue of origin is lung, while presence of APC and/or PIK3CA variants suggest that the tissue of origin is not lung. In general, detection of variants in genes including KEAP1, LRP1B, EGFR, IKZF1, ARID2, FAT1, GRM3, ERBB4, IL7R, BCORL1, ATM, SMAD4, KMT2C, PAK7, TET2, KDM6A, POLE, IRF4, ATR, KRAS, TAF, PMS1, CHEK2, SYK, NRAS, ALK, and POLD1 provide positive evidence for a lung tissue source of origin, while detection of variants in genes including APC and PIK3CA provide negative evidence for a lung tissue source of origin.

FIG. 6D depicts an example of model coefficient outputs for features associated with different genes, in relation to a prediction of a non-cancer grouping. For example, as shown in FIG. 6D, presence of TP53 variant (negative coefficient) strongly suggests cancer rather than non-cancer. It is noted that the positive coefficient gene variants in FIG. 6D (e.g., FANCL, HIST1H3I, RPS6KB2, PHOX2B) can be due to presence of contaminating samples in the non-cancer group that may really have cancer, and that improved clinical status would improve the training set. As shown in FIG. 6D, other gene variants indicative of cancer, in accordance with their negative coefficients, include PBRM1, ATR, ALK, STAG2, CTNNB1, MGA, KAT6A, KDR, SMAD4, ERBB4, PTPRT, ARID1A, EGFR, BRAF, NOTCH1, DNMT3A, CREBBP, APC, KMT2D, PIK3CA, KRAS, and LRP1B.

FIG. 6E depicts an example of model coefficient outputs for features associated with different genes, in relation to a prediction of a pancreas tissue source of origin. For example, as shown in FIG. 6E, KRAS variant is indicative that the tissue of origin is pancreas. In general, detection of variants in genes including KRAS, U2AF1, KMT2D, SMAD4, TGFBR1, FANCE, and TP53 provide positive evidence for a pancreas tissue source of origin, while detection of variants in genes including FLT4 and DNMT1 provide negative evidence for a pancreas tissue source of origin.

FIG. 6F depicts an example of model coefficient outputs for features associated with different genes, in relation to a prediction of a bladder tissue source of origin. As shown in FIG. 6F, JAK2, KDM6A, and ALOX12B gene variants have positive coefficients and provide positive evidence for a bladder tissue source of origin.

FIG. 6G depicts an example of model coefficient outputs for features associated with different genes in relation to a prediction of a cancer of unknown primary tissue source of origin. As shown in FIG. 6G, STK11, SMARCA4, KRAS, TP53, SPTA1, LRP1B, EPHA7, IDH1, and INPP4B gene variants have positive coefficients and provide positive evidence for a cancer of unknown primary tissue source of origin.

FIG. 6H depicts an example of model coefficient outputs for features associated with different genes in relation to a prediction of a cervical tissue source of origin. As shown in FIG. 6H, CCND3 and RFWD2 gene variants have positive coefficients and provide positive evidence for a cervix tissue source of origin.

FIG. 6I depicts an example of model coefficient outputs for features associated with different genes in relation to a prediction of an esophageal tissue source of origin. As shown in FIG. 6I, LRP1B, ERBB4, SPTA1, IGF1R, EGFR, SPEN, FGFR1, DOT1L, FYN, IGF1, RUNX1, FOXO1, PTCH1, AR, PTPRT, and ERCC3 gene variants have positive coefficients and provide positive evidence for an esophageal tissue source of origin.

FIG. 6J depicts an example of model coefficient outputs for features associated with different genes in relation to a prediction of a gastric tissue source of origin. As shown in FIG. 6J, KRAS, DNMT1, and PREX2 gene variants have positive coefficients and provide positive evidence for a gastric tissue source of origin.

FIG. 6K depicts an example of model coefficient outputs for features associated with different genes in relation to a prediction of a head and neck tissue source of origin. As shown in FIG. 6K, KLHL6, NOTCH1, PBRM1, PIK3CB, KMT2D, ZRSR2, HIST1H1C, SPTA1, NPM1, SMARCA4, B2M, and CTNNA1 gene variants have positive coefficients and provide positive evidence for a head and neck tissue source of origin.

FIG. 6L depicts an example of model coefficient outputs for features associated with different genes in relation to a prediction of a hepatobiliary tissue source of origin. As shown in FIG. 6L, CCNE1, PIK3C2G, CTNNB1, SLIT2, TSHR, TCF7L2, TGFBR2, and RPTOR gene variants have positive coefficients and provide positive evidence for a hepatobiliary tissue source of origin.

FIG. 6M depicts an example of model coefficient outputs for features associated with different genes in relation to a prediction of a lymphoma tissue source of origin. As shown in FIG. 6M, CREBBP, SOCS1, BCL2, KMT2D, PDGFRB, TNFRSF14, BCR, REL, and AMER1 gene variants have positive coefficients and provide positive evidence for a lymphoma tissue source of origin.

FIG. 6N depicts an example of model coefficient outputs for features associated with different genes in relation to a prediction of a melanoma tissue source of origin. As shown in FIG. 6N, DNMT3B and EPHA3 gene variants have positive coefficients and provide positive evidence for a melanoma tissue source of origin.

FIG. 6O depicts an example of model coefficient outputs for features associated with different genes in relation to a prediction of a multiple myeloma tissue source of origin. As shown in FIG. 6O, BRAF, FUBP1, IDH2, and IRF4 gene variants have positive coefficients and provide positive evidence for a multiple myeloma tissue source of origin.

FIG. 6P depicts an example of model coefficient outputs for features associated with different genes in relation to a prediction of a tissue source of origin considered as “other”, such as other cancer types not shown in FIGS. 6A-6U. As shown in FIG. 6P, PAX3, CXCR4, and KMT2C gene variants have positive coefficients and provide positive evidence for a tissue source of origin class of other.

FIG. 6Q depicts an example of model coefficient outputs for features associated with different genes in relation to a prediction of an ovarian tissue source of origin. As shown in FIG. 6Q, ATR, TP53, TNFRS14, FANCC, KLF4, MSH2, FAT1, and BRCA2 gene variants have positive coefficients and provide positive evidence for an ovarian tissue source of origin.

FIG. 6R depicts an example of model coefficient outputs for features associated with different genes in relation to a prediction of a prostate tissue source of origin. As shown in FIG. 6R, TBX3, GRIN2A, MGA, and SPEN gene variants have positive coefficients and provide positive evidence for a prostate tissue source of origin, while PTPRD, SPTA1, NOTCH1, KMT2D, PIK3CA, KMT2C, APC, LRP1B, and KRAS gene variants have negative coefficients and provide negative evidence for a prostate tissue source of origin.

FIG. 6S depicts an example of model coefficient outputs for features associated with different genes in relation to a prediction of a renal tissue source of origin. As shown in FIG. 6S, VHL, MST1R, IDH2, TSC1, NOTCH1, EP300, and SNCAIP gene variants have positive coefficients and provide positive evidence for a renal tissue source of origin.

FIG. 6T depicts an example of model coefficient outputs for features associated with different genes in relation to a prediction of a thyroid tissue source of origin. As shown in FIG. 6T, a BRAF gene variant has a positive coefficient and provides positive evidence for a thyroid tissue source of origin, while a TP53 gene variant has a negative coefficient and provides negative evidence for a thyroid tissue source of origin.

FIG. 6U depicts an example of model coefficient outputs for features associated with different genes in relation to a prediction of a uterine tissue source of origin. As shown in FIG. 6U, CDC73, SF3B1, PTEN, TET1, and EPHB1 gene variants have positive coefficients and provide positive evidence for a uterine tissue source of origin, while a TP53 gene variant has a negative coefficient and provides negative evidence for a uterine tissue source of origin.

In relation to outputting a prediction according to embodiments of method steps described herein, the processing system can generate a prediction of a tissue type as the tissue source of origin upon evaluating values of one or more of the set of features related to that tissue type. For example, for a certain tissue or cancer type, the processing system can evaluate one or more, two or more, three or more, four or more, five or more, eight or more, or ten or more, of any of the small variant features listed for that cancer type in FIGS. 6A-6U. In some examples, a gene panel (e.g., targeted sequencing panel for generating a prediction of the tissue type as the tissue source of origin) can include genes and/or gene features corresponding to the one or more, two or more, three or more, four or more, five or more, eight or more, or ten or more gene features listed in its corresponding tissue or cancer type at FIGS. 6A-6U. Still further, the tissue of origin assessment and/or gene panel (e.g., targeted gene panel) can generate predictions for any combination of the tissue source of origin listed above, by evaluating, for each tissue source of origin of interest, any combination of its one or more, two or more, three or more, four or more, five or more, eight or more, or ten or more gene features listed in its corresponding figure of FIGS. 6A-6U.

### 7. Example Computer System

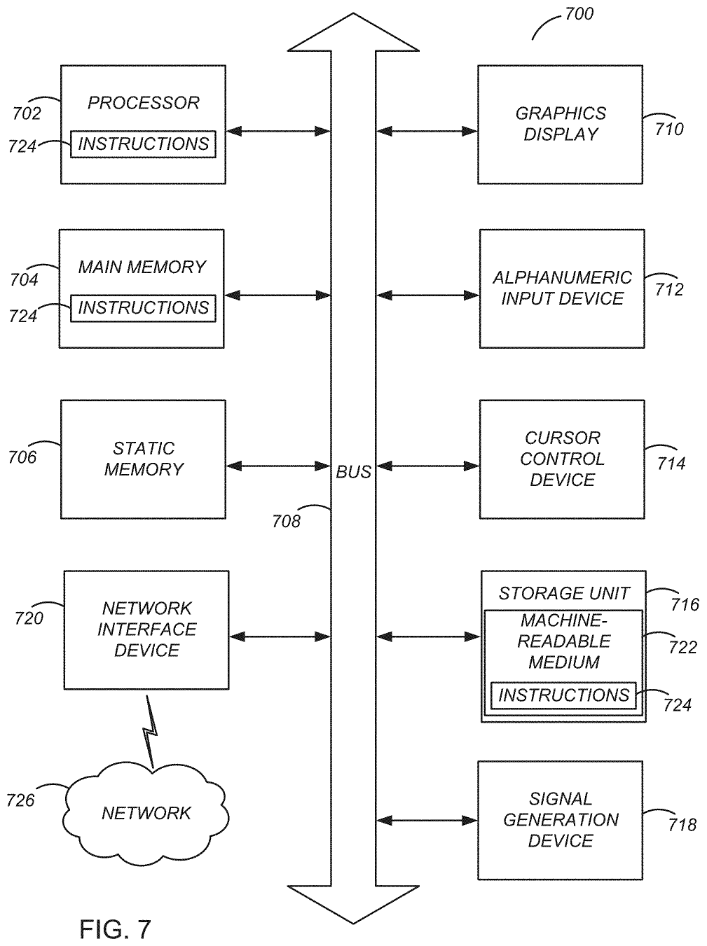

FIG. 7 shows a schematic of an example computer system for implementing various methods of the processes described herein, according to an embodiment. In particular, FIG. 7 is a block diagram illustrating components of an example computing machine that is capable of reading instructions from a computer-readable medium and executing them using a processor (or controller). A computer as described herein may include a single computing machine as shown in FIG. 7, a virtual machine, a distributed computing system that includes multiples nodes of computing machines shown in FIG. 7, or any other suitable arrangement of computing devices.

By way of example, FIG. 7 shows a diagrammatic representation of a computing machine in the example form of a computer system 700 within which instructions 724 (e.g., software, program code, or machine code), which may be stored in a computer-readable medium for causing the machine to perform any one or more of the processes discussed herein may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 7 may correspond to any software, hardware, or combined components (e.g., those shown in FIGS. 5A and 5B or a processing unit described herein), including but not limited to any engines, modules, computing server, machines that are used to perform one or more processes described herein. While FIG. 7 shows various hardware and software elements, each of the components described herein may include additional or fewer elements.

By way of example, a computing machine may be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 724 that specify actions to be taken by that machine. Further, while only a single machine is illustrated, the term “machine” and “computer” may also be taken to include any collection of machines that individually or jointly execute instructions 724 to perform any one or more of the methodologies discussed herein.

The example computer system 700 includes one or more processors 702 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 700 may also include a memory 704 that store computer code including instructions 724 that may cause the processors 702 to perform certain actions when the instructions are executed, directly or indirectly by the processors 702. Instructions can be any directions, commands, or orders that may be stored in different forms, such as equipment-readable instructions, programming instructions including source code, and other communication signals and orders. Instructions may be used in a general sense and are not limited to machine-readable codes.

One or more methods described herein improve the operation speed of the processors 702 and reduces the space required for the memory 704. For example, the machine learning methods described herein reduces the complexity of the computation of the processors 702 by applying one or more novel techniques that simplify the steps in training, reaching convergence, and generating results of the processors 702. The algorithms described herein also may reduce the size of the models and datasets to reduce the storage space requirement for memory 704.

The performance of certain of the operations may be distributed among the more than one processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations. Even though in the specification or the claims may refer some processes to be performed by a processor, this should be construed to include a joint operation of multiple distributed processors.

The computer system 700 may include a main memory 704, and a static memory 706, which are configured to communicate with each other via a bus 708. The computer system 700 may further include a graphics display unit 710 (e.g., a plasma display panel (PDP), a liquid crystal display (LCD), a projector, or a cathode ray tube (CRT)). The graphics display unit 710, controlled by the processors 702, displays a graphical user interface (GUI) to display one or more results and data generated by the processes described herein. The computer system 700 may also include alphanumeric input device 712 (e.g., a keyboard), a cursor control device 714 (e.g., a mouse, a trackball, a joystick, a motion sensor, or other pointing instrument), a storage unit 716 (a hard drive, a solid state drive, a hybrid drive, a memory disk, etc.), a signal generation device 718 (e.g., a speaker), and a network interface device 720, which also are configured to communicate via the bus 708.

The storage unit 716 includes a computer-readable medium 722 on which is stored instructions 724 embodying any one or more of the methodologies or functions described herein. The instructions 724 may also reside, completely or at least partially, within the main memory 704 or within the processor 702 (e.g., within a processor's cache memory) during execution thereof by the computer system 700, the main memory 704 and the processor 702 also constituting computer-readable media. The instructions 724 may be transmitted or received over a network 726 via the network interface device 720.

While computer-readable medium 722 is shown in an example embodiment to be a single medium, the term “computer-readable medium” should be taken to include a single non-transitory medium or multiple media (e.g., a centralized or distributed database, or associated caches and servers) able to store instructions (e.g., instructions 724). The computer-readable medium may include any medium that is capable of storing instructions (e.g., instructions 724) for execution by the processors (e.g., processors 702) and that cause the processors to perform any one or more of the methodologies disclosed herein. The computer-readable medium may include, but not be limited to, data repositories in the form of solid-state memories, optical media, and magnetic media.

### 8. Additional Considerations

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention can be changed without departing from the scope of the present invention. Furthermore, the foregoing description is for the purpose of illustration only, and not for the purpose of limitation.

1. A method for determining a cancer tissue of origin for a subject, the method comprising:
accessing, upon processing a cell-free deoxyribonucleic acid (cfDNA) sample from the subject, a dataset comprising sequence reads generated from application of a physical assay to the cfDNA sample;
performing a computational assay on the dataset to generate values of a set of features;
processing the set of features with a prediction model to generate a prediction of a cancer tissue of origin for the subject from a set of candidate tissue sources, the prediction model transforming the values of the set of features into the prediction through a function; and
returning the prediction of the cancer tissue of origin for the subject.

2. The method of claim 1, further comprising generating a value of a confidence parameter for the prediction and, upon determining satisfaction of a threshold condition by the value, providing the prediction to an entity.

3. The method of claim 1, wherein processing the set of features with the prediction model comprises:
classifying the subject into one of a cancerous group and a non-cancerous group upon applying a first sub-model of the prediction model, and
upon determining that the subject is classified into the cancerous group, applying a second sub-model of the prediction model to generate the prediction of the cancer tissue of origin for the subject.

4. The method of claim 3, further comprising: based upon an output of the first sub-model, performing a reflex assay on a reserve sample from the subject, and based upon the reflex assay, classifying the subject into one of the cancerous group and the non-cancerous group.

5. The method of claim 3, wherein the first sub-model is a binary classification model that allows for a non-negative coefficient output corresponding to increased likelihood of cancer classification.

6. The method of claim 3, wherein the first sub-model is a binary classification model that allows for a negative coefficient output corresponding to decreased likelihood of cancer classification.

7. The method of claim 5, wherein the binary classification model comprises an alpha parameter configured to tune performance of the first sub-model between a ridge-like regression mode and a lasso-like regression mode, the method further comprising evaluating a contribution of each of a set of small variant features to the prediction and adjusting the alpha parameter based upon the contributions.

8. The method of claim 5, wherein the binary classification model comprises a specificity condition characterizing cancer signal strength, and wherein determining that the subject is classified into the cancerous group comprises comparing a specificity value associated with the cfDNA sample to the specificity condition.

9. The method of claim 3, wherein an output set of coefficients of the first sub-model comprises a coefficient output corresponding to a first feature of the set of features, the first feature characterizing presence of a small variant in the cfDNA sample, and wherein processing the set of features comprises:
identifying, from the cfDNA sample, a signal corresponding to the first feature, and
classifying the subject into the cancerous group based on the magnitude of the coefficient output corresponding to the first feature.

10. (canceled)

11. The method of claim 3, wherein the second sub-model is a multinomial regression model, and wherein the prediction provided by the multinomial regression model comprises a set of values, each value indicating a probability that the cfDNA sample originated from one of the set of candidate tissue sources associated with that value.

12. The method of claim 11, wherein the multinomial regression model comprises an alpha parameter configured to tune performance of the second sub-model between a ridge-like regression mode and a lasso-like regression mode, the method further comprising evaluating a contribution of each of the set of small variant features to the prediction and adjusting the alpha parameter based upon the contributions.

13. The method of claim 3, wherein the second sub-model comprises at least one of:
a support vector machine comprising architecture for evaluating each of the set of candidate tissue sources against other candidate tissue sources of the set of candidate tissue sources;
a random forest classifier comprising learned weights derived from cfDNA samples of a population of subjects; and
a gradient boosting machine.

14.-15. (canceled)

16. The method of claim 1, wherein processing the set of features with a prediction model comprises:
applying a penalized multinomial regression model to the set of features, the penalized multinomial regression model comprising a set of functions with a set of coefficients trained by a dataset derived from cfDNA samples of a population of subjects satisfying a specificity condition that characterizes cancer signal strength, and the penalized multinomial regression model allowing negative coefficients.

17. The method of claim 16, wherein the penalized multinomial regression model allows for a negative coefficient output corresponding to decreased likelihood of classification to a first tissue source of the set of candidate tissue sources, a zero coefficient output corresponding to indeterminate classification, and a positive coefficient output corresponding to increased likelihood of classification to the first tissue source of the set of candidate tissue sources.

18. The method of claim 16,
wherein the set of coefficients of the penalized multinomial regression model comprises a negative coefficient corresponding to a first feature of the set of features, the first feature characterizing presence of a small variant in the cfDNA sample, and
wherein processing the set of features to generate the prediction of the cancer tissue of origin for the subject comprises:
identifying, from the cfDNA sample, a signal corresponding to the first feature, and excluding a candidate tissue source of the set of candidate tissue sources from the prediction based on the magnitude of the negative coefficient corresponding to the first feature.

19. The method of claim 16, wherein the set of coefficients of the penalized multinomial regression model comprises a positive coefficient corresponding to a second feature of the set of features, the second feature characterizing presence of a second small variant in the cfDNA sample, and wherein processing the set of small variant features to generate the prediction of the cancer tissue of origin for the subject comprises: identifying, from the cfDNA sample, a signal corresponding to the second feature, and outputting a candidate tissue source of the set of candidate tissue sources as the prediction based on the magnitude of the positive coefficient corresponding to the second feature.

20.-22. (canceled)

23. The method of claim 1, wherein processing the set of features with the prediction model comprises processing values of at least one small variant feature of a set of small variant features derived from application of a small variant assay on nucleic acids in the cfDNA sample, wherein the set of small variant features is:
a count of somatic variants;
a count of non-synonymous variants;
a count of variants per gene represented in the cfDNA sample;
an allele frequency for at least one variant;
a relative order statistics feature that represents a comparison of an allele frequency for a first variant to an allele frequency for at least one other variant;
a maximum variant allele frequency of a nonsynonymous variant associated with a gene;
a mutation interaction feature describing joint presence of a first mutation and a second mutation for one or more genes; or
an oncogenic-associated feature.

24.-33. (canceled)

34. The method of claim 1, wherein processing the set of features with the prediction model comprises processing values of at least one copy number feature of a set of copy number features derived from application of a copy number assay on nucleic acids in the cfDNA sample, the set of copy number features comprising at least one of:
a focal copy number of a mutation, the focal copy number describing repetition of a genetic variation represented in below a threshold proportion of a sequence from the cfDNA sample; and
features associated with at least one of fusions and structural variants.

35.-47. (canceled)

48. The method of claim 1, wherein generating a prediction of the cancer tissue of origin comprises evaluating values of the set of features corresponding to one or more of a set of small variant features listed in TABLES 3-22.

49.-67. (canceled)

68. A computer product comprising a non-transitory computer-readable medium storing a plurality of instructions for controlling a computer system to perform:
accessing, upon processing a cell-free deoxyribonucleic acid (cfDNA) sample from the subject, a dataset comprising sequence reads generated from application of a physical assay to the cfDNA sample;
performing a computational assay on the dataset to generate values of a set of features;
processing the set of features with a prediction model to generate a prediction of a cancer tissue of origin for the subject from a set of candidate tissue sources, the prediction model transforming the values of the set of features into the prediction through a function; and
returning the prediction of the cancer tissue of origin for the subject.
